(19) United States
(12) Patent Application Publication
(10) Pub. No.: US 2026/0007752 A1
(43) Pub. Date: Jan. 8, 2026

(54) HYDROLYTICALLY DEGRADABLE ZWITTERIONIC POLYMERS

(71) Applicant: University of Maryland, College Park, College Park, MD (US)

(72) Inventor: Alexander K. Andrianov, Gaithersburg, MD (US)

(21) Appl. No.: 19/261,887

(22) Filed: Jul. 7, 2025

Related U.S. Application Data
(60) Provisional application No. 63/668,149, filed on Jul. 5, 2024.

(51) Int. Cl. A61K 47/34; C08G 79/025

(52) U.S. Cl. CPC A61K 47/34; C08G 79/025

(57) ABSTRACT

Provided are polymers with one or more domains. At least one domain of the polymer is zwitterionic. The zwitterionic domain may be a polyphosphazene having one or more HEPES-based groups. In various examples, the polymer is zwitterionic. The repeat unit in polyphosphazenes has the following general formula:

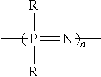

where n is an integer. Each R may be the same or different. The pendant groups are also referred to herein as R and R′. At least one of the zwitterionic group may have the following structure:

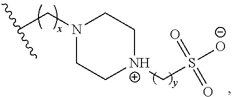

or a protonated or deprotonated form thereof. Also provided are methods of making and using the polymers of the present disclosure.

### CROSS REFERENCE TO RELATED APPLICATIONS

This application claims priority to U.S. Provisional Application No. 63/668,149, filed Jul. 5, 2024, the disclosure of which is hereby incorporated by reference.

### BACKGROUND OF THE DISCLOSURE

Zwitterionic macromolecules and polyampholytes-synthetic polymers carrying both positive and negative charges in their structures, have recently emerged as some of the most advanced systems for modulating bio-macromolecular interactions. Among the most important features of these polymers is their ability to introduce anti-fouling properties into surfaces of biomaterials or induce protein-repulsive stealth behavior in macromolecular therapies. Both of these capabilities are typically attributed to the formation of a strong hydration layer associated with amphoteric macromolecules, which provides resistance to a non-specific protein attachment. In case of biomedical devices and prostheses, this prevents the undesirable adsorption of proteins and cells on the surface—the first step in the initiation of serious adverse medical events, such as formation of biofilms or thrombosis. Similarly, the creation of a highly hydrated dynamic steric barrier around protein and peptide therapeutics reduces their undesirable recognition by the host immune system. This, in turn, minimizes their intrinsic immunogenicity and a rapid clearance—well-known shortcomings of advanced macromolecular drugs and nanomedicines.

Current strategies in the formation of hydrated steric (stealth) barriers on surfaces or around nanomedicines continue to rely on PEGylation—a commercially successful technology, which is based on covalent modification of a substrate with poly(ethylene glycol)(PEG). However, recent studies on the emergence of anti-PEG antibodies pose reasonable concerns over further advancement of this strategy. In fact, anti-PEG immune responses were reported to increase the risk of adverse reactions ranging from local inflammation to complement-mediated immediate type hypersensitivity, which may occasionally result in fatal anaphylaxis. Systematic studies suggested that the immunogenicity of PEG is linked to the intrinsic hydrophobic characteristics of this polymer and antibodies were described as “backbone” and “methoxy” specific. In a search for PEG-alternatives, zwitterionic macromolecules hold a significant potential due to their highly charged nature, which provides for a resistance to non-specific protein attachment. Comparison of zwitterionic polycarboxy betaine with PEG reveals its superior activity in the reduction of protein antigenicity and the absence of anti-polymer antibodies.

Molecular engineering of polymers bearing both negative and positive charges has emerged as a thriving field of study. However, the diversity of currently available structures remains largely insufficient to satisfy needs of life sciences applications. Moreover, their synthesis requires complex multi-step synthetic processes. Polyampholytes—the most developed family of macromolecules, in which oppositely charged groups are located in separate repeat units of the copolymer, are typically characterized by a difficult to control charge asymmetry. The scope of zwitterionic or inner salt structures implemented in the same pendant groups of polymers is strikingly narrow as a vast majority of such macromolecules have been built on the basis of polymethacrylates containing betaine functionalities. Furthermore, the existing water-soluble zwitterionic polymers are generally not fully biodegradable, which is a critical limitation for their use as part of injectable pharmaceutical formulations.

### BRIEF SUMMARY OF THE DISCLOSURE

Zwitterionic polymers—ampholytic macromolecules containing ionic moieties of opposite sign on the same pendant groups, exhibit strong protein-repulsive properties and an inherent biological inertness. For that reason, these highly hydrated inner salt macromolecules have emerged as some of the most viable alternatives to poly(ethylene glycol)(PEG)—a gold standard in enabling stealth behavior in life sciences applications. However, the structural diversity of polymer zwitterions remains limited and currently available macromolecules do not possess an intrinsic ability to undergo hydrolytical degradation—an important prerequisite for use in drug delivery applications. The present disclosure provides the synthesis of a zwitterionic polymer—a multimerized form of a widely used, biologically benign buffering agent—4-(2-hydroxyethyl)-1-piperazineethanesulfonic acid (HEPES), which is covalently assembled on a hydrolytically degradable polyphosphazene backbone. The resulting high molar mass polymer—polyHEPES-pz contains approximately two thousand pendent HEPES moieties per macromolecular chain and shows excellent solubility in aqueous solutions. Direct visualization of this polymer within the vitrified state using cryogenic electron microscopy (cryoEM) reveals individual chains of linear macromolecules. The polymer exhibits typical polyzwitterionic solution behavior, such as “salting-in” effect and upper critical solution temperature (UCST) type miscibility profile. Poly HEPES-pz displays pH and temperature dependent hydrolytic degradation pattern, demonstrates excellent in vitro compatibility with human red blood cells and strong resistance to interactions with plasma proteins-features, which highlight its potential utility for life sciences applications.

In an aspect, the present disclosure provides a polymer comprising one or more domains. At least one domain of the polymer is zwitterionic. The zwitterionic domain may be a polyphosphazene having one or more HEPES-based groups. In various examples, the polymer is zwitterionic.

Polyphosphazenes are polymers with backbones having alternating phosphorus and nitrogen, separated by alternating single and double bonds. Each phosphorous atom is covalently bonded to two pendant groups (“R”). The repeat unit in polyphosphazenes has the following general formula:

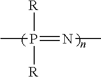

wherein n is an integer. Each R may be the same or different. The pendant groups are also referred to herein as R and R′.

In various examples, the at least one zwitterionic group has the following structure:

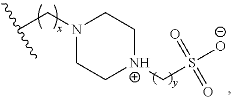

or a protonated or deprotonated form thereof, wherein x is 1 to 3 (e.g., 1, 2, or 3) and y is 1 to 3 (e.g., 1, 2, or 3). In various examples, the zwitterionic group has the following structure:

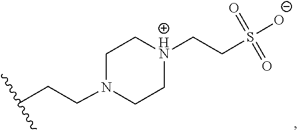

or a protonated or deprotonated form thereof. Such a group may be referred to as a “HEPES” group. In various example, a polymer of the present disclosure comprises at least one HEPES groups. In various examples, fewer than 1% of all R groups are HEPES groups. In various examples, 1 to 100% of all R groups are HEPES groups.

In various examples, the polymer has the following structure:

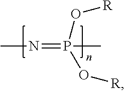

wherein every R group is:

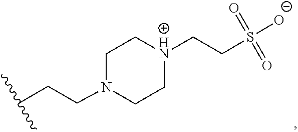

or a protonated or deprotonated form thereof, and n is 2 to 500,000. In such an example, the polymer only has one domain (i.e., the zwitterionic domain). Amino groups of the polymer can be quaternized by alkylation of tertiary amino groups using alkyl halides. In this case, the polymer may be a member of the polysulfobetaine family.

In an aspect, the present disclosure provides compositions comprising one or more polymers of the present disclosure. The compositions may further comprise one or more pharmaceutically acceptable carrier(s).

In an aspect, the present disclosure provides uses of polymer carriers of the present disclosure. For example, the carriers can be used to delivery one or more pharmaceutical agents to a subject.

For example, a method of delivering a pharmaceutical agent to an individual in need of a pharmaceutical agent comprising administering one or more compositions comprising one or more pharmaceutical agents and polymers of the present disclosure.

In an aspect, the present disclosure provides substrates having a polymer of the present disclosure disposed thereon. Examples of substrates include, but are not limited to, implants or prosthetics made from metal, plastic, ceramic or other materials, such as cardiac stents, cerebral spinal fluid shunt systems, cochlear implants, metal-on-metal hip implants, phakic intraocular lenses, surgical mesh used for hernia repair, urogynecologic surgical mesh implants, artificial joints, breast implants, bone, muscle, and joint fusion hardware. The substrates having a polymer disposed thereon may have antifouling properties.

### BRIEF DESCRIPTION OF THE FIGURES

For a fuller understanding of the nature and objects of the disclosure, reference should be made to the following detailed description taken in conjunction with the accompanying figures.

FIG. 1A. Chemical structures and schematic representation of zwitterionic HEPES buffering agent and its macromolecular multimer-polyphosphazene containing zwitterionic HEPES as pendent groups (polyHEPES-pz) with polyphosphazene backbone, cationic and anionic moieties highlighted.

FIG. 1B. Synthetic pathway to polyHEPES-pz: (i) preparation of dodecyltrimethylammonium (DDTMA) salt of HEPES, (I) macromolecular substitution of PDCP in the presence of triethylamine (TEA) and (II) deprotection of the substituted polyphosphazene intermediate to yield poly HEPES-pz.

FIG. 2A. The appearance of polyHEPES-pz as a solid and in aqueous solution.

FIG. 2B. Size-exclusion chromatography profile of polyHEPES-pz (mass average molar mass shown, 230 nm, 2 mg/mL polymer, 100 mM phosphate buffer, pH 7.4).

FIG. 2C. Dynamic light scattering (DLS) profiles of polyHEPES-pz in aqueous solutions of various pH values (distribution by intensity, 2 mg/mL polymer).

FIG. 2D. A representative cryoEM image of a vitrified sample of polyHEPES-pz and projection of its trajectory.

FIG. 3A. 1H NMR spectra of polyHEPES-pz at various pH.

FIG. 3B. Protonation-deprotonation scheme for polymer side groups.

FIG. 3C. Dependence of 1H NMR chemical shifts of poly HEPES-pz on pH.

FIG. 3D. comparison of 1H NMR chemical shifts for poly HEPES (diamonds) and HEPES (triangles) at pH 14 showing a downfield shift of peaks (blue arrows) for the polymer.

FIG. 4A. Hydrodynamic diameter (DLS, 2 mg/mL polyHEPES-pz).

FIG. 4B. Zeta-potential (0.1 mg/mL polyHEPES-pz).

FIG. 4C. UV absorbance at 190 nm and 202 nm (1 mg/mL polyHEPES-pz) as a function of aqueous solution pH values (pKa₁ and pKa₂—estimated negative logarithms of dissociation constants for sulfonic acid and piperazine moieties of poly HEPES-pz, correspondingly)

FIG. 5A. Hydrodynamic diameter of polyHEPES-pz in an aqueous solution as a function of sodium chloride concentration demonstrating a “salting-in” effect (z-average diameters shown, 2 mg/mL poly HEPES-pz, pH 7.4)

FIG. 5B. Hydrodynamic diameter of polyHEPES-pz in an aqueous solution in a salt-free solution as a function of temperature showing agglomeration at lower temperatures (z-average diameters shown, 1 mg/mL polyHEPES-pz, deionized water, pH 7.4).

FIG. 6A. Dependencies of a residual molar mass of polyHEPES-pz on time at various incubation temperatures (SEC, 100 mM phosphate buffer, pH 7.4).

FIG. 6B. SEC profiles at various incubation times (37° C., 100 mM phosphate buffer, pH 7.4).

FIG. 6C. Dependencies of residual molar mass of polyHEPES-pz on time in aqueous solutions of various pH values (SEC, 37° C., 100 mM phosphate buffer).

FIG. 7A. Comparison of hemolytic activity of polyHEPES with PCPP, PEG and PEI (numbers indicate the percent of hemolysis, 0.25 mL of 0.05 mg/mL polymers, 0.25 mL of 10 mg/mL human red blood cells, PBS, pH 7.4, incubation at 37° C. for 3 h).

FIG. 7B. AF4 fractograms of plasma, poly HEPES-pz and their mixture.

FIG. 7C. AF4 fractograms of polyHEPES-pz, HSA and their mixture (2.5 mg/mL polyHEPES-pz: 0.05 mg/mL HSA: reconstituted human plasma was subjected to 790-fold dilution: PBS, pH 7.4).

FIG. 8A. 13C NMR spectra of polyHEPES-pz: (a) full and (b) zoomed-in version (D₂O, 25 mg/mL polyHEPES-pz, pD 7.4, 100 MHz).

FIG. 8B. PolyHEPES-pz ¹³C NMR chemical shifts: 61.75, 57.34, 52.21, 52.07, 51.44, 47.71 ppm (HEPES 13C NMR chemical shifts (for comparative purposes): 61.82, 57.29, 52.22, 51.95, 51.43, 47.71 ppm).

FIG. 9A. ¹H NMR spectra of polyHEPES-pz: full spectrum at pH 7.4

FIG. 9B. ¹H NMR spectra of poly HEPES-pz: zoomed-in versions of polymer solutions at pH 14 (400 MHZ, D₂O, 25 mg/mL polyHEPES-pz).

FIG. 9C. ¹H NMR spectra of poly HEPES-pz: zoomed-in versions of polymer solutions at pH 7.4 (400 MHZ, D₂O, 25 mg/mL polyHEPES-pz).

FIG. 9D. ¹H NMR spectra of polyHEPES-pz: zoomed-in versions of polymer solutions at pH 5 (400 MHZ, D₂O, 25 mg/mL polyHEPES-pz).

FIG. 10A. ¹H NMR spectra of HEPES in solutions of different pH values.

FIG. 10B. pH-dependence of chemical shifts (10 mg/mL HEPES, D₂O, pH of solutions was adjusted by adding 0.5 M sodium hydroxide or 0.5 M hydrochloric acid in D₂O, water peak (4.8 ppm) was used as an internal standard, 400 MHZ).

FIG. 11A. 31P NMR spectra of polyHEPES-pz in solutions of different pH values (161.9 MHZ, D₂O).

FIG. 11B. Potential patterns of side groups linked to phosphorus atoms, which can result from different protonation-deprotonation states of piperazine groups.

FIG. 12A. Representative cryoEM images of a vitrified sample of polyHEPES-pz showing single polymer chains.

FIG. 12B. Histogram of polymer size distribution on the basis of cryoEM images.

FIG. 12C. Examples of cryoEM images that were used for plotting size distribution histogram (0.1 mg/mL polyHEPES-pz, 5% sodium chloride, pH 7.4).

FIG. 13. UV absorbance spectra of polyHEPES-pz in solutions at different pH values (1 mg/mL polyHEPES-pz).

FIG. 14. DLS profiles of polyHEPES-pz in aqueous solutions in the presence of various concentrations of sodium chloride demonstrating a “salting-in” effect (2 mg/mL poly HEPES-pz. pH 7.4).

FIG. 15. DLS profiles of polyHEPES-pz in salt-free aqueous solutions at various temperatures showing bimodal size distribution at lower temperatures (1 mg/mL polyuHEPES-pz, deionized water, pH 7.4).

FIG. 16. Degradation of poly HEPES-pz as monitored by 31P NMR (60° C., pH 5.0).

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, process step, and electronic changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%: 0.5% to 2.4%: 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

The phrase “therapeutically effective amount” is used herein to mean an amount sufficient to reduce by at least about 15 percent, preferably by at least 50 percent, more preferably by at least 90 percent, and most preferably prevents oxidative stress in the individual. Alternatively, a therapeutically effective amount is sufficient to cause an improvement in a clinically significant condition in the individual.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

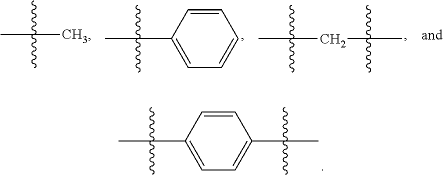

As used herein, unless otherwise indicated, the term “aliphatic” or “aliphatic groups” refers to branched or unbranched hydrocarbon groups that, optionally, contain one or more degree(s) of unsaturation. Degrees of unsaturation can arise from, but are not limited to, cyclic aliphatic groups. For example, the aliphatic groups/moieties are a C₁₆ to C₄₀ aliphatic group, including all integer numbers of carbons and ranges of numbers of carbons therebetween (e.g., C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). Aliphatic groups include, but are not limited to, alkyl groups, alkene groups, and alkyne groups. The aliphatic group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxy late groups, carboxylic acid groups, ether groups, ester groups, amide groups, thioether groups, thioester groups, and the like, and combinations thereof.

As used herein, unless otherwise indicated, the term “alkyl” or “alkyl group” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₄₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). The alkyl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, the term “cycloalkyl” or “cycloalkyl group” refers to a cyclic hydrocarbon group, e.g., cyclopropyl, cyclobutyl, cyclohexyl, and cyclopentyl groups. Cycloalkyl groups can be saturated or partially unsaturated ring systems optionally substituted with, for example, one to three substituents. Each substituent is independently chosen from alkyl, —NH₂, oxo (═O), phenyl, haloalkyl (e.g., —CF₃), halo (e.g., —F, —Cl, —Br, —I), alkoxy, and —OH groups. Additionally, alkyl substituents may be substituted with various other functional groups. Additional non-limiting examples include aliphatic groups (e.g., alkyl groups, alkenyl groups, alkynyl groups, and the like), halogenated aliphatic groups (e.g., trifluoromethyl group), aryl groups, halogenated aryl groups, alkoxide groups, nitro groups, carboxylate groups, carboxylic acids, ether groups, alkyne groups (e.g., acetylenyl groups and the like), and the like, and combinations thereof.

As used herein, unless otherwise indicated, the term “aryl” or “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxy benzyl groups, tolyl groups, xylyl groups, and the like.

As used herein, the term “heteroaryl” or “heteroaryl group” refers to a monocyclic or bicyclic ring system comprising one or two aromatic rings and containing at least one nitrogen or oxygen atom in an aromatic ring. Unless otherwise indicated, a heteroaryl group can be unsubstituted or substituted with one or more, and in particular one or two, substituents. Non-limiting examples of substituents include halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Examples of heteroaryl groups include, benzofuranyl, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl groups, and substituents analogs of any of the foregoing heteroaryl groups.

As used herein, unless otherwise indicated, the term “alkoxy” or “alkoxy group” refers to

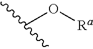

where R^{a} is a linear, branched or cyclic C₁-C₆ alkyl group, including all integer numbers of carbons and ranges of numbers of carbons therebetween. For example, suitable alkoxy groups include methoxy, ethoxy, propoxy, iso-propoxy, butoxy, sec-butoxy, tert-butoxy, and hexoxy groups. Additionally, alkyl substituents can be substituted with various other functional groups, e.g. functional groups disclosed herein.

As used herein, unless otherwise indicated, the term “amino” or “amino group” refers to

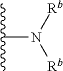

where each R^{b} is selected independently from the group consisting of hydrogen atom, substituted or unsubstituted C₁-C₁₀ alkyl, including all integer numbers of carbons and ranges of numbers of carbons therebetween, substituted or unsubstituted phenyl, substituted or unsubstituted heteroaryl, substituted carbonyl, substituted sulfonyl, haloalkyl, and substituted or unsubstituted benzyl groups.

As used herein, unless otherwise indicated, the term “benzyl” or “benzyl group” refers to

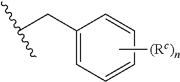

where R^{c} is a substituent on the phenyl ring and n is from 0 to 5. The substituents can be the same or different. For example, the substituents on the benzyl group include substituted or unsubstituted alkyl, —NH₂, phenyl, haloalkyl (e.g., —CF₃), halo (e.g., —F, —Cl, —Br, —I), alkoxy (e.g., —OMe), and —OH groups.

As used herein, unless otherwise indicated, halogen means fluorine, chlorine, bromine, and iodine, and halo means fluoro, chloro, bromo, and iodo.

As used herein, unless otherwise indicated, the term “phenoxy” or “phenoxy group” (—OPh) refers to

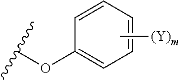

where each Y is independently selected from the group consisting of F, Cl, Br, and I and m can be 0, 1 or 2.

As used herein, unless otherwise indicated, the term “phenyl” or “phenyl group” means

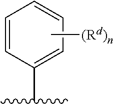

where each R^{d} is an independent substituent on the phenyl group and n is from 0 to 5. The substituents at different occurrences can be the same or different. For example, the substituents on the phenyl group include substituted or unsubstituted C₁-C₆ alkyl, including all integer numbers of carbons and ranges of numbers of carbons therebetween, substituted or unsubstituted amino, haloalkyl (e.g., —CF₃), halo (e.g., —F, —Cl, —Br, —I), substituted or unsubstituted alkoxy (e.g., —OMe), and sulfonyl group. In certain instances, two adjacent R groups can be connected through to form a dioxolyl group.

Zwitterionic polymers—ampholytic macromolecules containing ionic moieties of opposite sign on the same pendant groups, exhibit strong protein-repulsive properties and an inherent biological inertness. For that reason, these highly hydrated inner salt macromolecules have emerged as some of the most viable alternatives to poly(ethylene glycol)(PEG)—a gold standard in enabling stealth behavior in life sciences applications. However, the structural diversity of polymer zwitterions remains limited and currently available macromolecules do not possess an intrinsic ability to undergo hydrolytical degradation—an important prerequisite for use in drug delivery applications. The present disclosure provides the synthesis of a zwitterionic polymer—a multimerized form of a widely used, biologically benign buffering agent—4-(2-hydroxyethyl)-1-piperazineethanesulfonic acid (HEPES), which is covalently assembled on a hydrolytically degradable polyphosphazene backbone. The resulting high molar mass polymer—polyHEPES-pz contains approximately two thousand pendent HEPES moieties per macromolecular chain and shows excellent solubility in aqueous solutions. Direct visualization of this polymer within the vitrified state using cryogenic electron microscopy (cryoEM) reveals individual chains of linear macromolecules. The polymer exhibits typical polyzwitterionic solution behavior, such as “salting-in” effect and upper critical solution temperature (UCST) type miscibility profile. PolyHEPES-pz displays pH and temperature dependent hydrolytic degradation pattern, demonstrates excellent in vitro compatibility with human red blood cells and strong resistance to interactions with plasma proteins-features, which highlight its potential utility for life sciences applications.

In an aspect, the present disclosure provides a polymer comprising one or more domains. At least one domain of the polymer is zwitterionic. The zwitterionic domain may be a polyphosphazene having one or more HEPES-based groups. In various examples, the polymer is zwitterionic.

Polyphosphazenes are polymers with backbones having alternating phosphorus and nitrogen, separated by alternating single and double bonds. Each phosphorous atom is covalently bonded to two pendant groups (“R”). The repeat unit in polyphosphazenes has the following general formula:

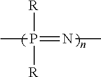

wherein n is an integer. Each R may be the same or different. The pendant groups are also referred to herein as R and R′.

In various examples, the polymer comprises the following structure:

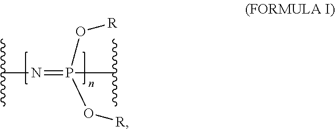

where each R group may be the same or different, and at least one R group of the polymer is a zwitterionic group or capable of being ionized such that the R group is zwitterionic. Non-zwitterionic R groups may be substituted or unsubstituted aliphatic groups, which may be or comprise linear aliphatic chains, branching aliphatic chains, and/or cyclic aliphatic groups or substituted or unsubstituted aryl groups. In various examples, the aliphatic group may comprise an aryl group. In various examples, the aliphatic group may have one or more fluoro groups. In other examples, the R groups may be groups capable of binding pharmaceutical agents. n is an integer and is from 1 to 500,000 (e.g., 3 to 500,000). The polymer comprising FORMULA I wherein at least one R group is a zwitterionic group or capable of being ionized such that the R group is zwitterionic may be referred to as a the zwitterionic domain. Additionally, as used throughout, the term “zwitterionic” can be used to describe groups that are zwitterionic and groups that can be protonated or deprotonated such that they become a zwitterionic species.

In various examples, R groups that are not zwitterionic or capable of being ionized such that they are zwitterionic groups may be aliphatic and comprise various substituents. Examples of substituents are described herein. Non-limiting examples include, but are not limited to, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, amino groups, sulfhydryl groups, imidazolyl groups, phenolic groups, guanidyl groups, indolyl groups, ether groups, ester groups, amide groups, thioether groups, thioester groups, and the like, and combinations thereof. For example, one or more aliphatic R groups may be trifluoroethoxy groups. R groups may further comprise an ionizable substituent. R groups comprising ionizable substituents (that will not ionize to a zwitterionic state), may be referred to as “ionizable groups.”

In various examples, R groups that are aryl groups may be substituted or unsubstituted. The aryl groups may be phenyl, biphenyl, or like, wherein the aryl group can be further substituted with one or more aliphatic groups (e.g., alkyl groups, alkene groups, alkyne groups, and the like), aryl groups, hydroxyl groups, alkoxide groups, carboxylate groups, carboxylic acid groups, amino groups, sulfhydryl groups, imidazolyl groups, phenolic groups, guanidyl groups, indolyl groups, ether groups, ester groups, amide groups, thioether groups, thioester groups, and the like, and combinations thereof. For example, an R group that is an aryl group may have the following structure:

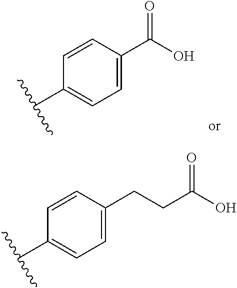 [SMILES: *c1ccc(C(=O)O)cc1 ; *c1ccc(CCC(=O)O)cc1]

or the like.

Various groups comprising ionizable groups may be used. For example, the group may be an aliphatic group that comprises an ionizable group or an aromatic group that comprises an ionizable group. Examples of such groups include, but are not limited to, -phenylSO₃H, -phenylPO₃H, -(aliphatic)CO₂H, -(aliphatic)SO₃H, -(aliphatic)PO₃H, -phenyl(aliphatic)CO₂H, -phenyl(aliphatic) SO₃H, -phenyl(aliphatic)PO₃H, -[(CH₂)ₓO]_{y}phenylCO₂H, -[(CH₂)ₓO]_{y}phenylSO₃H, -[(CH₂)ₓO]_{y}phenylPO₃H, -[(CH₂)ₓO]_{y} (aliphatic)CO₂H, -[(CH₂)ₓO]; (aliphatic)SO₃H, -[(CH₂)ₓO]_{y}(aliphatic)PO₃H, -[(CH₂)ₓO]_{y}phenyl(aliphatic)CO₂H, -[(CH₂)ₓO]_{y}phenyl(aliphatic)SO₃H, or -[(CH₂)ₓO]_{y}phenyl(aliphatic)PO₃H, and deprotonated analogs thereof, where the aliphatic group may be a C₁ to C₈ aliphatic group (e.g., C₁ to C₈ alkyl group), x is an in integer from 1 to 8, and y is an integer from 1 to 20. Additional examples of groups comprising ionizable groups include but are not limited to -(aliphatic)N(CH₃)₂, -(aliphatic)N(C₂H₅)₂, -(aliphatic)NH₂, -(aliphatic)NH(CH₃), -(aromatic)N(CH₃)₂, -(aromatic)N(C₂H₅)₂, -(aromatic)NH₂, -(aromatic)NH(CH₃), quaternary ammonium groups, heterocyclic amines, alkylimidazoles, pyridines, pipyridines, diamines, allylamines, quinolines, isoquinolines, benzoquinolines, imidazoquinolines, polyamines, various amino acids, peptides, aminosaccharides, and ammonium salts thereof, where the aliphatic group may be a C₁ to C₈ aliphatic group (e.g., C₁, C₂, C₃, C₄, C₅, C₆, C₇, or C₈ alkyl group) or the aromatic group may be a C₅ to C₁₆ group (e.g., C₁, C₂, C₃, C₄, C₅, C₆, C₇, or C₈, C₉, C₁₀, C₁₁, C₁₂, C₁₃, C₁₄, C₁₅, or C₁₆ alkyl group).

Polymers of the present disclosure can include ligands. Polymers of the present disclosure can bind pharmaceutical agents or to enable intermolecular interactions. The binding ligands of the present disclosure include functionalities capable of forming covalent or non-covalent attachments with a therapeutic drug or nanoparticulate delivery vehicles. For example, covalent bonds may be formed. For example, non-covalent interactions, such as, for example, Coulombic interactions, x-x interactions, cationic-x interactions, hydrophobic interactions, and the like, and combinations thereof.

In a various examples, the ligands can include functional groups R suitable for covalent attachment of drug, such as, for example: amino groups for conjugation reactions using N-hydroxysuccinimide (NHS) esters, imidoester, hydroxymethyl phosphine, guanidination, fluorophenyl esters, carbodiimides, anhydrides, arylating agents, carbonates, aldehydes, and glyoxals: carboxylate groups for conjugation reactions using carbodiimides: thiol groups for reactions with maleimide, haloacetyl, pyridyldisulfide, vinyl sulfone: hydroxyl groups for conjugation reactions using isocyanates, carbonyldiimidazole: aldehyde and ketone groups for conjugation reactions using hydrazine derivative, Schiff base formation, and reductive amination.

In various examples, a polymer may comprise one or more R group comprising one or more carboxylate groups. In various examples, the carboxylates may be suitable for conjugation to a drug. The drug may be reacted with the carboxylated via acylation chemistry using an activating agent, such as, for example, ethyl-3-[3-dimethylaminopropyl]carbodiimide hydrochloride (EDC) with or without N-hydroxysuccinimide (NHS) or sulfo-N-hydroxysuccinimide (sulfo-NHS), diazomethane, diazoacetate esters and diazoacetamides. In yet another example, the carboxylate group of the copolymer is carboxylatoethylphenoxy group

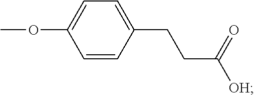

or carboxylatophenoxy group

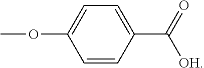

In various examples, the drug is the protein.

In various examples, the binding of a polymer to a therapeutic drug is through non-covalent interactions, such as electrostatic, hydrogen bonds, van der Waals forces, and hydrophobic effects. In such case the carrier forms a complex with a drug typically through a spontaneous self-assembly with drug in aqueous solutions.

In various examples, drug and polymer carrier binding is enabled through the establishment of multivalent interactions, such as ionic, hydrogen bond, receptor-ligand, host-guest inclusion, and peptide-protein interactions. Multivalent interactions are a desirable way to achieve effective binding, especially when individual binding interactions are weak. Multivalent interactions are also desirable when ‘flexible’ binding is important between the carrier and the protein drug allowing for the polymer ligand to jump from one binding site to another across a protein surface through a combination of mechanisms that can be likened to “hopping, walking, and flying.”

Examples of suitable ligands for multivalent interactions may include ionized carboxyl and tertiary amino groups, hydroxyl, carbonyl, non-ionized carboxyl groups, components of β-cyclodextrin-adamantane pair, pseudorotaxane pairs, such as α-cyclodextrin-poly(ethylene glycol), α-cyclodextrin-N-alkylpyridinium, and various complexes of cucurbit[n]urils with positively charged hydrophobic guests. Yet another example of possible ligands includes short, disordered peptide stretches, which partially mimic the interface area (pockets) of protein drugs. This can be represented by the binding of tyrosyl-phosphorylated peptides to proteins containing Src homology domain 2 (SH2) or phosphotyrosyl binding domain (PTB) domain, binding of peptides with certain proline motifs to proteins containing Src homology domain 3 (SH3).

In yet another example, binding ligands can contain hydrophobic alkyl groups to provide for interactions with poorly soluble drugs.

In various examples, the at least one zwitterionic group has the following structure:

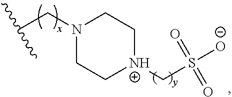

or a protonated or deprotonated form thereof, wherein x is 1 to 3 (e.g., 1, 2, or 3) and y is 1 to 3 (e.g., 1, 2, or 3). In various examples, the zwitterionic group has the following structure:

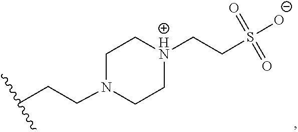

or a protonated or deprotonated form thereof. Such a group may be referred to as a “HEPES” group. In various example, a polymer of the present disclosure comprises at least one HEPES group. In various examples, fewer than 1% of all R groups are HEPES groups. In various examples, 1 to 100% of all R groups are HEPES groups.

In various examples, the polymer has the following structure:

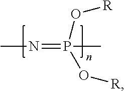

wherein every R group is:

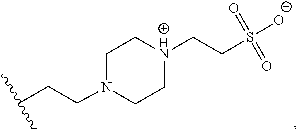

or a protonated or deprotonated form thereof, and n is 2 to 500,000. In such an example, the polymer only has one domain (i.e., the zwitterionic domain). Amino groups of the polymer can be quaternized by alkylation of tertiary amino groups using alkyl halides. In this case, the polymer may be a member of the polysulfobetaine family.

A polymer of the present disclosure may have various properties. For example, the polymer may have a molecular weight of 1 kDa to 10,000 kDa. The most preferred molecular weight is between 100 kDa and 1,000 kDa. The polymer may have a hydrodynamic diameter in a non-agglomerated state of 10 nm to 800 nm. The polymer may have a dispersity of 1 to 10 (e.g., 1, 2, 3, 4, 5, 6, 7, 8, 9, or 10). The polymer may have linear, branched, star-shaped, comb, dendritic or macrocyclic (ring polymer) topology.

In various examples, a single repeat unit of a polymer of the present disclosure may have the following structure:

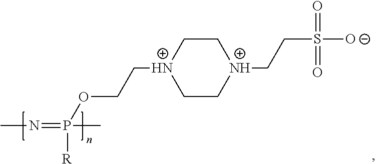

where R is described as herein, and protonated and deprotonated analogs thereof.

A polymer of the present disclosure may have desirable features. For example, a polymer of the present disclosure has a degradation rate that is dependent on pH. For example, when the polymer is a polyphosphazene where all the side chains are HEPES groups are resistant to degradation at a pH above 8 and are more prone to degradation below 6. Thus, without intending to be bound by any particular theory, it is considered that the degradation rate of the polymer may be tuned by varying the number of HEPES groups present in the polymer or adding hydrolytically labile or hydrolytically stable side groups. Additionally, the degradation rate of the polymers may also be temperature dependent. For example, the polymer may degrade faster at higher temperatures (e.g., above 30° C.), but may be more resistant to degradation at lower temperatures (e.g., less than 0° C.).

In various examples, the polymer of the present disclosure is a copolymer comprising one or more additional side groups or one or more additional side chains. In various examples, the copolymer may be a block copolymer, alternating copolymer, or a random copolymer. The macromolecular topology may include linear, macrocyclic, graft, star-, brush- or comb-shaped architectures.

In an aspect, the present disclosure provides compositions comprising one or more polymers of the present disclosure. The compositions may further comprise one or more pharmaceutically acceptable carrier(s).

The compositions may include one or more pharmaceutically acceptable carriers. Non-limiting examples of compositions include solutions, suspensions, emulsions, solid injectable compositions that are dissolved or suspended in a solvent before use, and the like. Injections may be prepared by dissolving, suspending, or emulsifying one or more of the active ingredients in a diluent. Non-limiting examples of diluents include distilled water (e.g., for injection), physiological saline, vegetable oil, alcohol, and the like, and combinations thereof. Injections may contain, for example, stabilizers, solubilizers, suspending agents, emulsifiers, soothing agents, buffers, preservatives, and the like, and combinations thereof. Injections may be sterilized in the final formulation step or prepared by sterile procedure. A pharmaceutical composition of the disclosure may also be formulated into a sterile solid preparation, for example, by freeze-drying, and may be used after sterilized or dissolved in sterile injectable water or other sterile diluent(s) immediately before use. Additional examples of pharmaceutically acceptable carriers include, but are not limited to, sugars, such as, for example, lactose, glucose, and sucrose: starches, such as, for example, corn starch and potato starch: cellulose, such as, for example, sodium carboxymethyl cellulose, ethyl cellulose, and cellulose acetate: powdered tragacanth: malt: gelatin: talc: excipients, such as, for example, cocoa butter and suppository waxes: oils, such as, for example, peanut oil, cottonseed oil, safflower oil, sesame oil, olive oil, corn oil, and soybean oil: glycols, such as, for example, propylene glycol: polyols, such as, for example, glycerin, sorbitol, mannitol, and polyethylene glycol: esters, such as, for example, ethyl oleate and ethyl laurate: agar; buffering agents, such as, for example, magnesium hydroxide and aluminum hydroxide: alginic acid: pyrogen-free water: isotonic saline: Ringer's solution: ethyl alcohol: phosphate buffer solutions; other non-toxic compatible substances employed in pharmaceutical formulations, and the like, and combinations thereof. Non-limiting examples of pharmaceutically acceptable carriers are found in: Remington: The Science and Practice of Pharmacy (2012) 22nd Edition, Philadelphia, PA. Lippincott Williams & Wilkins.

Compositions of the disclosure can comprise more than one pharmaceutical agent. For example, a first composition comprising a compound of the disclosure, and a first pharmaceutical agent can be separately prepared from a composition which comprises the same compound of the disclosure and a second pharmaceutical agent, and such preparations can be mixed to provide a two-pronged (or more) approach to achieving the desired prophylaxis or therapy in an individual. Further, compositions of the disclosure can be prepared using mixed preparations of any of the compounds disclosed herein.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Various antioxidants may be used. Examples of antioxidants include: (1) water soluble antioxidants, such as ascorbic acid, cysteine hydrochloride, sodium bisulfate, sodium metabisulfite, sodium sulfite and the like: (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In various examples, there is no covalent bond between the pharmaceutical agent and the polymer. In an example, a pharmaceutical agent (e.g., a small molecule, antibiotic, immunomodulatory compound, nucleic acid, peptide or protein) is not a hydrophobic pharmaceutical agent. In an embodiment, a pharmaceutical agent (e.g., a small molecule, antibiotic, immunomodulatory compound, nucleic acid, peptide or protein) is a water-soluble pharmaceutical agent.

In an example, pharmaceutical agents are small molecules. In an example, the pharmaceutical agents are antibiotics and immunomodulatory compounds. In another example, pharmaceutical agent are nucleic acids. In the most preferred embodiment, pharmaceutical agent are protein or peptide drugs. A pharmaceutical agent can be any pharmaceutical agent used for therapy of, for example, cancers, immune disorders, infections, and other diseases.

Examples of protein drugs include, but not limited to antibody-based drugs, Fc fusion proteins, anticoagulants, blood factors, bone morphogenetic proteins, engineered protein scaffolds, enzymes, cytokines, growth factors, hormones, interferons, interleukins, and thrombolytics.

In another example, the pharmaceutical agents are monoclonal antibodies (MAbs), which include, but not limited to abciximab, rituximab, basiliximab, palivizumab, infliximab, trastuzumab, alemtuzumab, adalimumab, tositumomab-I131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab, brentuximab. In the most preferred embodiment, protein drugs are bispecific Mabs, including, but not limited to bi-specific T-cell engagers (BiTEs) and Dual-Affinity Re-Targeting (DART) mabs.

Examples of nucleic acid drugs include DNA-Based Therapeutics, such as, for example, oligonucleotides for antisense and antigene Applications, Aptamers, DNAzymes and RNA-Based Therapeutics, such as, for example, RNA Aptamers, RNA Decoys, Antisense RNA, Ribozymes, Small Interfering RNAs (siRNAs), and MicroRNA.

Examples of peptide drugs include, but not limited to hormones, neurotransmitters, growth factors, ion channel ligands, and anti-infectives. They include GLP-1 aganists, such as, for example, Byetta™ (exenatide), Bydureon™ (exenatide), Victoza™ (liraglutide), Lyxumia™ (lixisenatide), and most recently Tanzeum™ (albiglutide), Cpd86, ZPGG-72, MOD-6030, ZP2929, HM12525A, VSR859, NN9926, TTP273/TTP054, ZYOGI, MAR709, TT401, HM11260C, PB1023, Dulaglutide, Semaglutide, ITCA. Multifunctional peptides can include a hybrid of two peptides being bound together like modules either directly or via a linker, conjugates with small molecules, oligoribonucleotides, or antibodies.

Example of small drugs include poorly water-soluble drugs. Suitable poorly water soluble pharmaceutical agents include, but are not limited to, taxanes (such as, for example, paclitaxel, docetaxel, ortataxel and other taxanes), epothilones, camptothecins, colchicines, geladanamycins, amiodarones, thyroid hormones, amphotericin, corticosteroids, propofol, melatonin, cyclosporine, rapamycin (sirolimus) and derivatives, tacrolimus, mycophenolic acids, ifosfamide, vinorelbine, vancomycin, gemcitabine, thiotepa, bleomycin, polymyxin, and diagnostic radiocontrast agents.

A composition comprising one or more pharmaceutical agents of the present disclosure can be water-soluble. For example, a composition comprising one or more pharmaceutical agents of the present disclosure can provide a homogenous aqueous solution.

In an aspect, the present disclosure provides uses of polymer carriers of the present disclosure. For example, the carriers can be used to delivery one or more pharmaceutical agents to a subject.

For example, a method of delivering a pharmaceutical agent to an individual in need of a pharmaceutical agent comprising administering one or more compositions comprising one or more pharmaceutical agents and polymers of the present disclosure.

In various examples, disclosure comprises administering a therapeutically effective amount of a composition described herein. The term “therapeutic” as used herein means a treatment and/or prophylaxis. The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound or composition that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. Compositions of the disclosure can be administered in conjunction with any conventional treatment regimen, including sequential or simultaneous administration of other agent(s) that are intended to treat or prevent a disease or disorder.

The individual to be treated by the method of the disclosure may be human or non-human (e.g., mammal). Non-human animals include ungulates such as bovines. Additional on-limiting examples of non-human mammals include pigs, mice, rats, rabbits, cats, dogs, or other agricultural mammals, pet, or service animals, and the like.

Administration of formulations/compositions of the present disclosure as described herein can be carried out using any suitable route of administration known in the art. For example, the compositions/compositions can be administered via intravenous, intramuscular, intraperitoneal, intracerebral, subcutaneous, intra-articular, intrasynovial, oral, topical, intradermal or inhalation routes. The compositions may be administered parenterally or enterically. In one example, it can be administered intradermally using microneedle patches. The compositions may be introduced as a single administration or as multiple administrations or may be introduced in a continuous manner over a period of time. For example, the administration(s) can be a pre-specified number of administrations or daily. weekly or monthly administrations, which may be continuous or intermittent, as may be clinically needed and/or therapeutically indicated.

In an aspect, the present disclosure provides substrates having a polymer of the present disclosure disposed thereon. Examples of substrates include, but are not limited to, implants or prosthetics made from metal, plastic, ceramic or other materials, such as cardiac stents, cerebral spinal fluid shunt systems, cochlear implants, metal-on-metal hip implants, phakic intraocular lenses, surgical mesh used for hernia repair, urogynecologic surgical mesh implants, artificial joints, breast implants, bone, muscle, and joint fusion hardware. The substrates having a polymer disposed thereon may have antifouling properties.

In an example, polymers of the present disclosure may be a portion or part of biocompatible materials. Polymers of the present disclosure can form the entire material, form blends with other materials, or they can be localized on the surface as a biocompatible coating. The material or its coating can include biologically active agents, which can be bound to the polymer or entrapped therein. When such agents are bound to the polymer of the present disclosure, they can constitute a side group covalently linked to the phosphazene backbone or associated with the polymer via ionic or hydrogen bonds. Alternatively, they can be bound to the polymer through hydrophobic or dipole-dipole interactions. In one embodiment, such biologically active agents can include anti-proliferative agents, such as sirolimus (rapamycin), gemcitabine, and paclitaxel. In a preferred embodiment, the polymer contains 4-methylumbelliferone (hymecromone) as a side group. In yet another embodiment the coating can also include antiplatelet agents, anticoagulants, anti-inflammatory agents, hypolipidemic agents, angiotensin-converting enzyme inhibitors, calcium antagonists and antioxidants. Coatings comprising polymers of the present disclosure can be applied to the material using a plurality of methods, such as spin-coating, 3-D printing, electrospinning, electrophoretic deposition, dip coating, thermal spray coating, plasma spray coating, pulsed laser deposition, roll coating and flow coating. In a preferred embodiment, the coating is applied from an aqueous solution using layer-by-layer technique. It can be applied using partner polyelectrolytes or hydrogen bond forming polymers or using poly HEPES polymer alone. When polymers of the present disclosure used in the layer-by-layer assembly in the absence of other polyelectrolytes or multifunctional small molecules, such as spermine or spermidine, they are applied sequentially as solutions of different pH.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

The following Statements provide various examples of the present disclosure. They are not intended to be limiting in any way.
Statement 1. A polymer comprising the following structure:

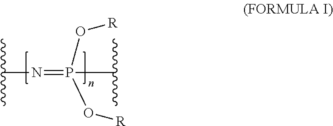

wherein each R group may be the same or different, each R group is individually a substituted or unsubstituted aliphatic group or substituted or unsubstituted aryl group, wherein at least one R group of the polymer is a zwitterionic group or capable of being ionized such that the R group is zwitterionic, and n is 1 to 500,000.
Statement 2. A polymer according to Statement 1, wherein the at least one R group has the following structure:

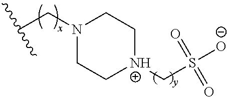

or a protonated or deprotonated form thereof, wherein x is 1 to 3 (e.g., 1, 2, or 3) and y is 1 to 3 (e.g., 1, 2, or 3).
Statement 3. A polymer according to Statement 1 or Statement 2, wherein the at least one R group is

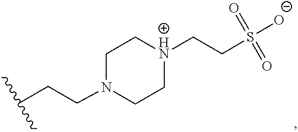

or a protonated or deprotonated form thereof.
Statement 4. A polymer according to any one of the preceding Statements, wherein 1 to all R groups are

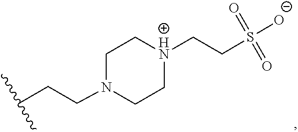

or a protonated or deprotonated form thereof.
Statement 5. A polymer according to any one of the preceding Statements, wherein the polymer is a copolymer comprising additional polymer units.
Statement 6. A polymer according to Statement 6, wherein the copolymer is a block copolymer.
Statement 7. A polymer according to Statement 5, wherein the copolymer is a random copolymer.
Statement 8. A polymer according to any one of the preceding Statements, wherein the R groups comprise one or more of the following substituents carboxylic acid groups, carboxylate groups, sulfonic acid groups, sulfonate groups, hydroxyl groups, oxy groups, ammonium groups, amino groups, sulfhydryl groups, imidazolyl groups, phenolic groups, guanidyl groups, indolyl groups or the like.
Statement 9. A polymer according to any one of the preceding Statements, wherein R is

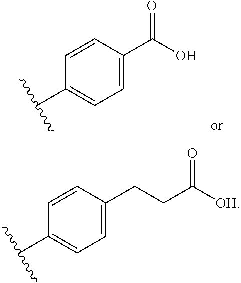 [SMILES: *c1ccc(C(=O)O)cc1 ; *c1ccc(CCC(=O)O)cc1]

Statement 10. A polymer according to any one of the preceding Statements, wherein the polymer has the following structure:

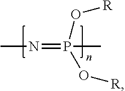

wherein every R group is:

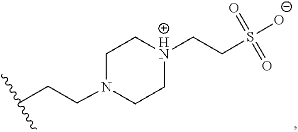

or a protonated or deprotonated form thereof, and n is 3 to 500,000.
Statement 11. A polymer according to any one of the proceeding Statements, wherein the polymer has a molecular weight of 1 kDa to 1,000 kDa.
Statement 12. A composition comprising one or more polymers according to any one of the preceding Statements.
Statement 13. A composition according to Statement 12, further comprising a pharmaceutically acceptable carrier.
Statement 14. A composition according to Statement 12 or Statement 13, further comprising one or more pharmaceutical agent.
Statement 15. A composition according to Statement 14, wherein the pharmaceutical agent is chosen from proteins, antibodies, peptides, enzymes, nucleic acids, drugs, small molecules, siRNA, and the like and combinations thereof.
Statement 16. A method of delivering a pharmaceutical agent to a subject in need of treatment comprising administering a composition according to Statement 14 to the subject.
Statement 17. A substrate wherein a polymer according to any one of Statements 1-11 is disposed thereon.
Statement 18. A substrate according to Statement 17, wherein the substrate is a medical device or prosthetic.

The following example is presented to illustrate the present disclosure. It is not intended to be limiting in any way.

### Example

This example provides description of the polyphosphazenes of the present disclosure.

The present disclosure provides the synthesis of polyHEPES-pz—a hydrolytically degradable water-soluble homopolymer containing HEPES as a side group. The synthetic pathway, which includes multimeric assembly of a buffering agent on a hybrid organic-inorganic polyphosphazene backbone, is enabled by a non-covalent protection of sulfonic acid functionalities. The resulting polymer, which contains approximately two thousand copies of HEPES, shows linear chain structure and typical zwitterionic solution behavior. PolyHEPES-pz exhibits important biophysical characteristics—pH and temperature dependent degradation profile, excellent hemocompatibility and protein repulsive properties—critical prerequisites for its utility in life sciences applications.

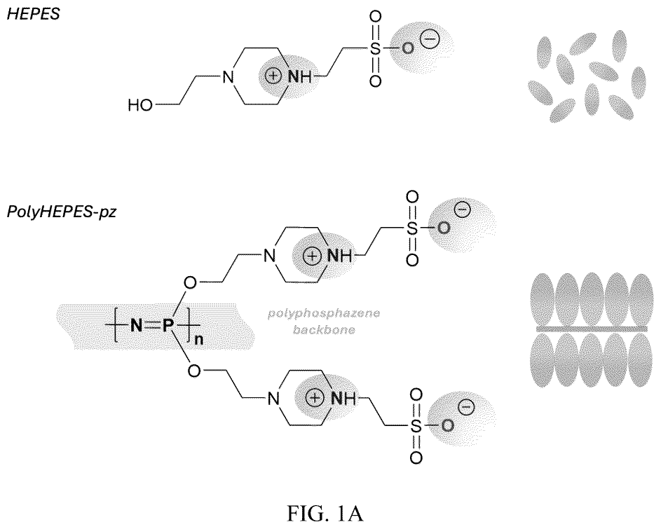

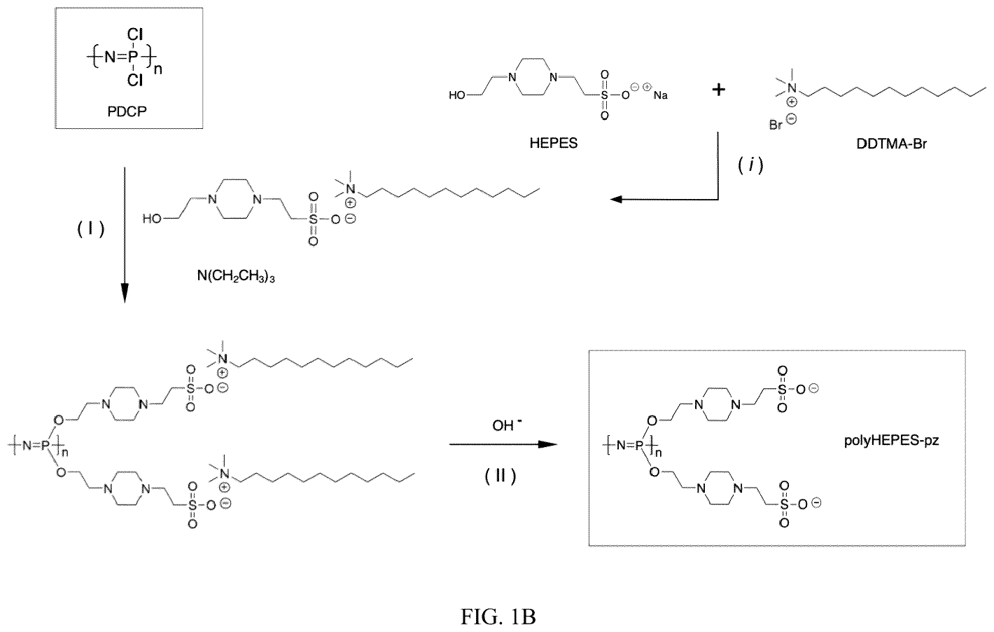

HEPES (4-(2-hydroxyethyl)-1-piperazineethanesulfonic acid (FIG. 1A)), is a widely used Good's buffer with a pH range corresponding to the physiological environment and pKa₂ (negative logarithm of the dissociation constant of the piperazine moiety) equals to approximately 7.5. The molecule is frequently referred to as a zwitterionic structure, as although not all moieties are permanently charged in the whole pH range, it bears both anionic and cationic charges at physiologically relevant pH values. The clinical safety of HEPES can be illustrated by its inclusion in the Inactive Ingredient in Approved Drug Product database of U.S. Food and Drug Administration (FDA) for intravenous administration with a maximum dose exposure of 41 mg. The underlying ampholytic nature of HEPES and its safety makes it an attractive candidate as a pendant group in a zwitterionic polymer intended for biomedical applications, especially if its backbone allows for biodegradation with the release of side groups. Prior attempts to incorporate HEPES in macromolecular structures using polyacrylate backbones were undertaken, but failed to produce water-soluble homopolymer, which was attributed to attractive interchain dipole-dipole interactions.

Materials. Diglyme (anhydrous, 99.5%), chlorobenzene (anhydrous, 99.8%), triethylamine. TEA (99.5%), potassium hydroxide, human serum albumin. HSA (≥96%)(Sigma-Aldrich. St. Louis, MO), dodecyltrimethyl ammonium bromide. DDTMA (Spectrum Chemical, Gardena, CA), hexachlorocyclotriphosphazene (Fushimi Pharmaceutical, Kagawa, Japan), 4-(2-hydroxyethyl)-1-piperazineethanesulfonic acid, HEPES (99%)(TCI, Portland, OR), phosphate buffered saline, PBS, methanol (HPLC grade) (ThermoFisher Scientific, Waltham, MA), ethanol (200 proof, anhydrous)(The Warner-Graham Company. Cockeysville. MD) and acetonitrile (HPLC grade)(Beantown Chemical Corporation. Hudson. NH) were used as received. Polydichlorophosphazene (PDCP) was synthesized via ring-opening polymerization of hexachlorocyclotriphosphazene in the titanium pressure reactor as described previously.

Preparation of Dodecyltrimethyl Ammonium Salt of HEPES, DDTMA-HEPES. A solution of HEPES-Na salt (5.91 g. 22.70 mmol in 10 mL deionized water) was adjusted to pH 7.5 using 2M sodium hydroxide and was added dropwise to an aqueous solution of DDTMA-Br (7 g. 22.70 mmol in 15 mL of deionized water). The mixture was kept at ambient temperature under stirring for 2 h and then lyophilized.

Synthesis of Poly[di(sulfonatoethanepiperazineethoxy) phosphazene], PolyHEPES-pz. All procedures were carried out under an atmosphere of dry nitrogen using MBraun Labstar Pro glovebox workstation (M. Braun Inertgas-Systeme GMBH, Garching, Germany) or common air-free laboratory techniques. HEPES-DDTMA (34.31 g, 60.34 mmol) was dissolved in 400 mL of anhydrous chlorobenzene and then TEA (6.53 g. 64.66 mmol) was added dropwise. The mixture was stirred for 30 min at ambient temperature under nitrogen and then brought to 70° C. Solution of PDCP (1 g. 8.62 mmol) in 20 mL of diglyme was then added dropwise to the mixture upon stirring. The reaction continued at this temperature for 24 h. After that period, aqueous solution of potassium hydroxide (10 mL, 13M) was added, and the mixture was stirred for an additional 1 h. The reaction mixture was then cooled to the ambient temperature and precipitate was collected by decantation. The precipitate was dissolved in a deionized water and purified by precipitations in methanol (twice) and ethanol. The resulting precipitate was re-dissolved in water and lyophilized to yield 1.7 g (65.35%) of the polymer.

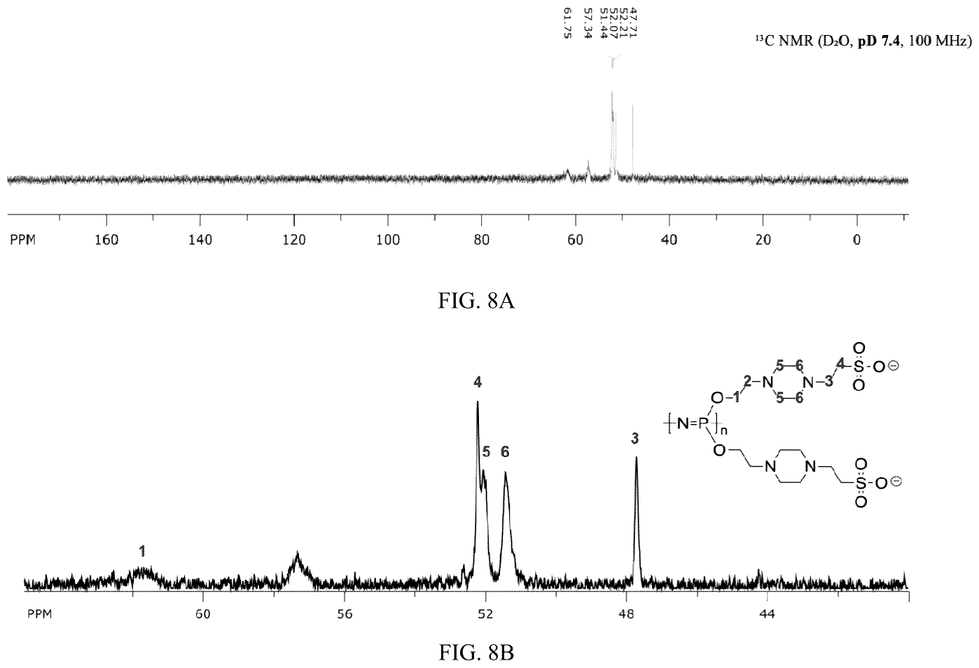

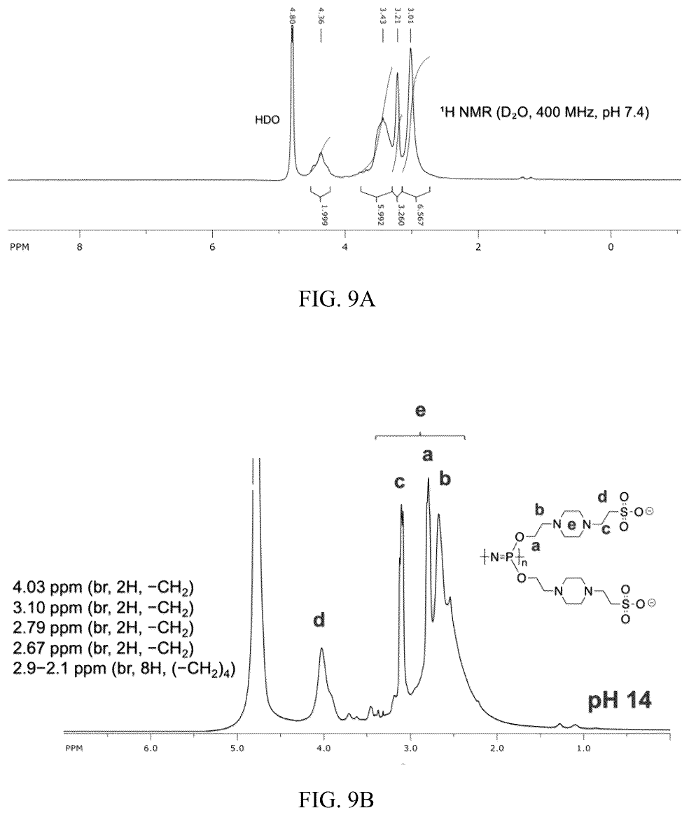

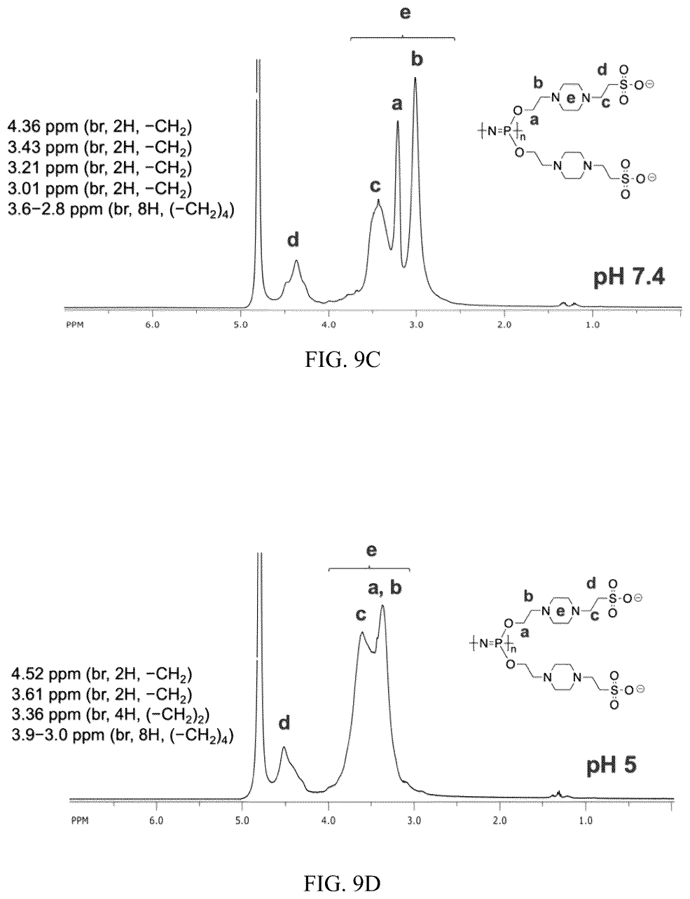

NMR spectra of polyHEPES-pz and ¹H NMR of HEPES (for comparison) are shown in FIGS. 8-11.

NMR Spectroscopy. ¹H NMR. ³¹C NMR and ³¹P NMR spectra were recorded using a 400 MHZ Ascend Bruker NMR spectrometer equipped with 400 MHZ magnet (Bruker Biospin Corp. Billerica. MA). Solutions of different pH were prepared by dissolving the polymer in deionized water followed by adjusting pH using sodium hydroxide or hydrochloric acid to a desired pH. The samples were then lyophilized and redissolved in D₂O to record spectra. The chemical shifts are as follows: ¹H NMR (400 MHZ, D₂O, pH 7.4). δ [ppm]: 4.36 ppm (br. 2H, —CH₂), 3.43 ppm (br. 2H, —CH₂), 3.21 ppm (br. 2H, —CH₂), 3.01 ppm (br. 2H, —CH₂), 3.6-2.8 ppm (br. 8H, (—CH₂)₄). ¹³C NMR (100 MHZ. D₂O), δ [ppm]: 61.75, 57.34, 52.21, 52.07, 51.44, 47.71 ppm. ³¹P NMR (161.9 MHZ. pH 14). δ [ppm]: −3.24.

Dynamic Light Scattering (DIS) and Z-potential Measurements. DLS studies and zeta-potential measurements were conducted using a Malvern Zetasizer Nano ZS instrument with data recorded and analyzed using Malvern Zetasizer 7.10 software (Malvern Instruments Ltd., Worcestershire. U.K.). Samples were filtered using Millex 0.22 mm filters prior to the analysis. Zeta-potential measurements were conducted using the same instrument by titrating polyHEPES-pz with 1.0 M hydrochloric acid.

Size-Exclusion Chromatography (SEC). Analysis of molar masses and polymer degradation studies were conducted using an Agilent 1260 Infinity II Binary LC system equipped with a G7112B binary pump. G7167A Multisampler. G7116A multicolumn thermostat. G7117C diode array detector. G7121A fluorescence detector (Agilent Technologies. Santa-Clara. CA) and TSKgel GMPW size-exclusion column (Tosoh Bioscience, LLC. Tokyo, Japan). PBS (0. 1×, pH 7.4) with 10% (v/v) acetonitrile was used as a mobile phase. Molar masses were calculated using poly(ethylene oxide) molar mass standards (American Polymer Standards Corporation. Mentor, OH).

UV-Vis Spectroscopy Measurements. UV-Vis spectra were recorded using a NanoDrop2000 spectrophotometer (Thermo Scientific. Wilmington. DE) in a wavelength range between 190 and 840 nm. The pH measurements were performed using Mettler Toledo pH-meter (Mettler-Toledo LLC. Oakland. CA). The pKₐ was obtained by determining the pH of the intersection of two absorption curves corresponding to maximum absorption at basic pH and the lowest reading wavelength at 190 nm for acidic pH.

Cryogenic Electron Microscopy (CryoEM). A sample droplet of aqueous polymer solution was deposited on holey carbon film TEM grids (Q3100CR1.3-2 nm. Electron Microscopy Sciences, Hatfield, PA), which were plasma-treated beforehand using glow discharge PELCO EasiGlow (Ted Pella Inc., Redding, CA). The grids were then double blotted on the Vitrobot (Vitrobot Mark IV, FEI, Hillsboro, OR) and vitrified in liquid ethane. The samples then were transferred to 200 kV Talos Arctica (FEI, Hillsboro, OR) equipped with FEI Falcon3EC direct electron detector. Imaging was performed at temperature about 90 K at an acceleration voltage of 200 kV. The data were collected using EPU software and processed in CryoSPARC 4.2.1 (Structura Biotechnology Inc., Toronto, Canada).

Hemolysis Assay. Hemolytic activity of polyHEPES-pz was evaluated using hemolysis test with human red blood cells (RBCs). Single donor human RBCs (Innovative Research, Inc., Novi. MI) were diluted with PBS to a hemoglobin concentration of 10 mg/mL. A hemoglobin human lyophilized powder (Sigma-Aldrich, St. Louis, MO) was used as a standard. Polymer solution (0.25 mL. 0.05 mg/mL in PBS) was mixed with RBCs (0.25 mL. 10 mg/mL) and incubated at 37° C. with agitation for 3 h. Cells were separated by centrifugation (14 000 rpm: 5 min), and the absorbance of the supernatant at 410 nm was recorded (Multiskan Spectrum microplate spectrophotometer. Thermo Fisher Scientific. Waltham, MA). Total hemoglobin concentration in samples before incubation was determined assuming that 100% lysis was achieved by diluting samples with ultrapure water. The percent of hemolysis was calculated as a ratio between the hemoglobin concentration in samples after and before incubation of RBC with test articles. All tests were conducted in triplicates. PEG with a molar mass of 8 kDa (Thermo Fisher Scientific, Waltham, MA) and poly[di(carboxylatophenoxy)phosphazene](PCPP) with a molar mass of 800 kDa were used for comparison. Polyethylenimine hydrochloride (PEI) with molar mass of 20 kDa was employed as a positive control.

Interactions of PolyHEPES-pz with Plasma Proteins. Lyophilized human plasma (Sigma. P9523-1 ML) was reconstituted in 1 mL deionized water and filtered using 0.2 μm filter. Plasma solution (0.25 mL) was diluted to match 0.05 mg/mL concentration of HSA. The AF4 studies were performed using AF2000 AT instrument (Postnova Analytics GmbH. Germany) equipped with the PN7149 solvent organizer. PN7520 solvent degasser. PN1130 isocratic focus pump. PN1130 isocratic tip pump. AF2000 module with two cross flow pumps. SPD-20A Prominence UV/VIS detector and a 10 kDa cutoff regenerated cellulose membrane (Postnova Analytics GmbH. Germany). PBS was employed as an eluent.

Synthetic Pathway to PolyHEPES-pz. Direct route to the synthesis of polyphosphazenes containing sulfonic acid moieties is hindered by a competitive reaction of polydichlorophosphazene (PDCP) with sulfonic acidic functionality of the nucleophilic reagent. Since the resulting P—O—S link is unstable, the reaction leads to a breakdown of the macromolecular chain. Modification of sulfonic acid functionalities with quaternary ammonium salts containing long alkyl chains provided for an adequate protection and allowed successful synthesis of polysulfonates. However, the efficiency of such non-covalent protection in case of zwitterionic molecules, such as HEPES remains unexplored. In particular, the release of hydrochloric acid, which accompanies reactions of PDCP, may cause protonation of piperazine rings of HEPES and weaken protection due to repulsive interactions between zwitterion and quaternary ammonium salt.

The overall synthetic pathway to polyHEPES-pz is shown in FIG. 1B. It involves preparation of quaternary ammonium salt of HEPES in aqueous solution (i), macromolecular substitution of PDCP with this reagent in organic phase (I) and removal of protective groups of the polymer (II). Testing of various quaternary ammonium salts resulted in the selection of dodecyltrimethyl ammonium (DDTMA) as it allows for an optimal balance between the solubility of HEPES derivative in the reaction mixture, efficient protection, and ease of protective group removal. The use of triethylamine (TEA) as an acceptor of hydrochloric acid, is essential to force the completion of the reaction and maintain adequate protection of sulfonic acid functionalities. The removal of protective groups after completion of the substitution, is facilitated by the addition of base, which causes precipitation of the polymer and its efficient isolation from the reaction system. The purification process of polyHEPES-pz, which includes repeated precipitations using water (solvent)-alcohol (non-solvent) pair, proved to be adequate for the ultimate removal of protective groups.

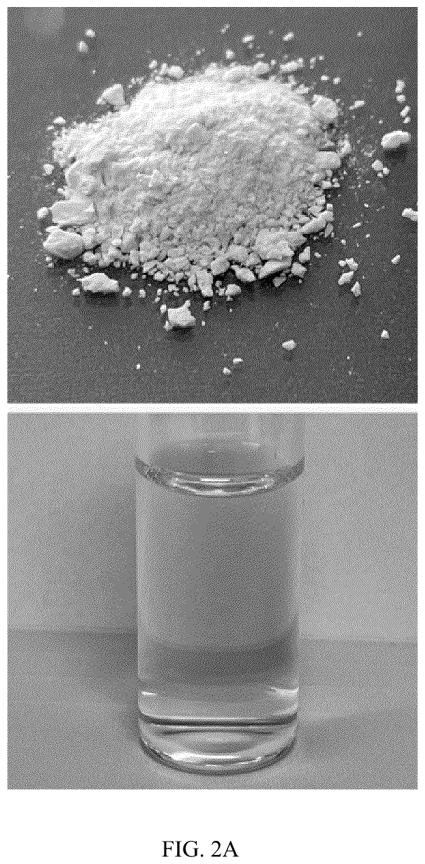

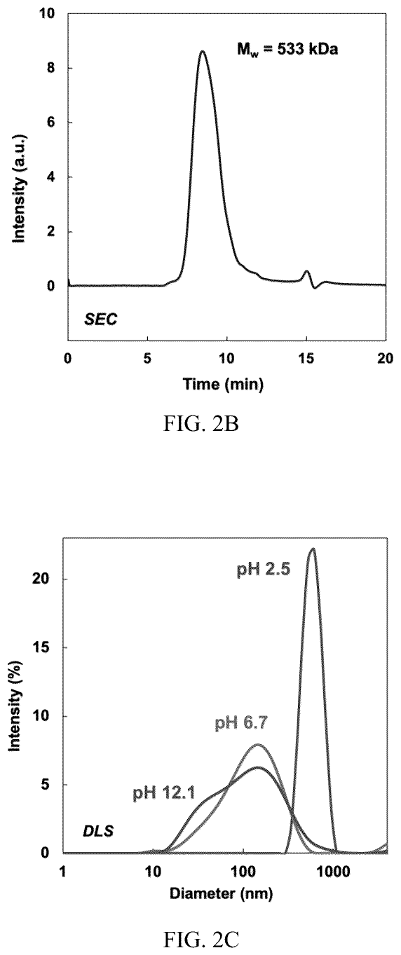

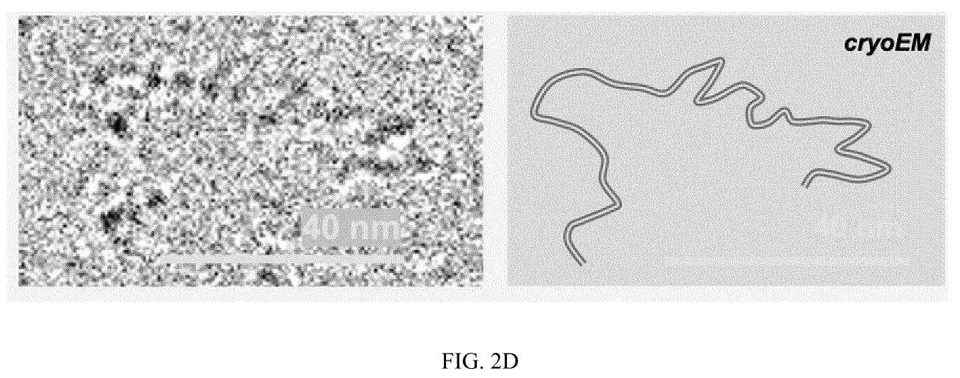

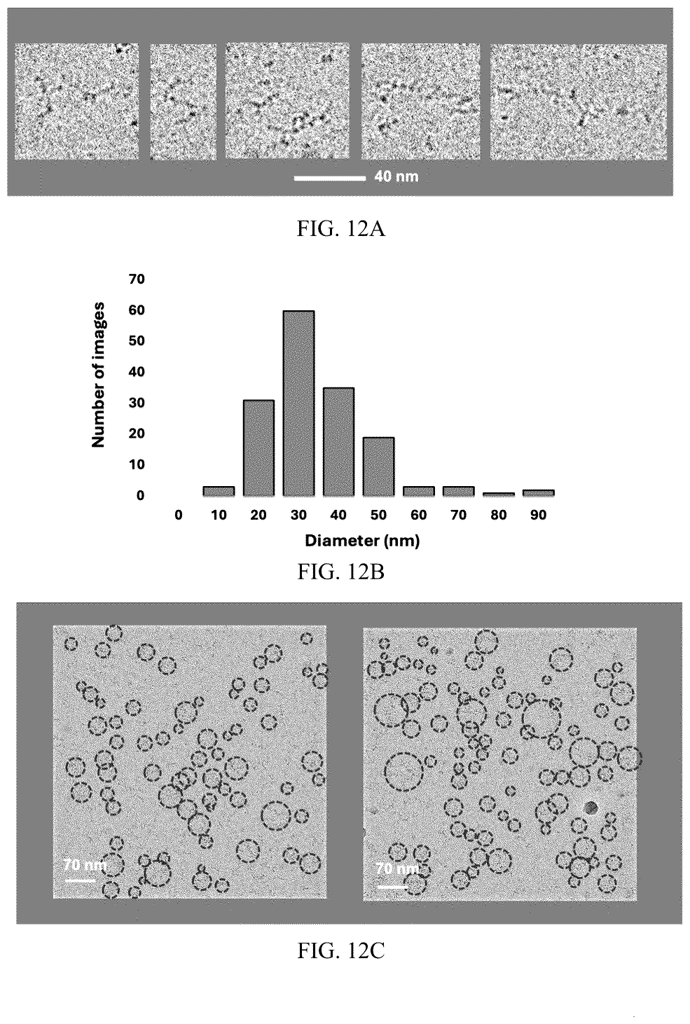

The resulting polyHEPES-pz is a white powdery substance with a yellow tint. which is practically insoluble in organic solvents, but forms clear solutions in water (FIG. 2A). The macromolecular nature of the polymer was confirmed by SEC and DLS analysis. SEC characterization of the polymer (FIG. 2B) reveals a unimodal molar mass distribution (mass average/number average values−533 kDa/230 kDa, dispersity−2.3). Based on the structure of a repeat unit, such molar mass corresponds to an estimated 2.000 HEPES molecules linked to a single polymer chain. DLS profile of polyHEPES-pz in PBS, pH 7.4 confirms the unimodal distribution with a z-average hydrodynamic diameter of 51 nm and polydispersity of 0.45 but is pH sensitive (FIG. 2C). Recent studies demonstrated that the mass contrast of inorganic phosphazene backbone allows direct visualization of single polyphosphazene chains by cryogenic electron microscopy (cryoEM). The cryoEM images of vitrified polyHEPES-pz samples can be described as random coils of linear polymer chains (FIG. 2D and 12).

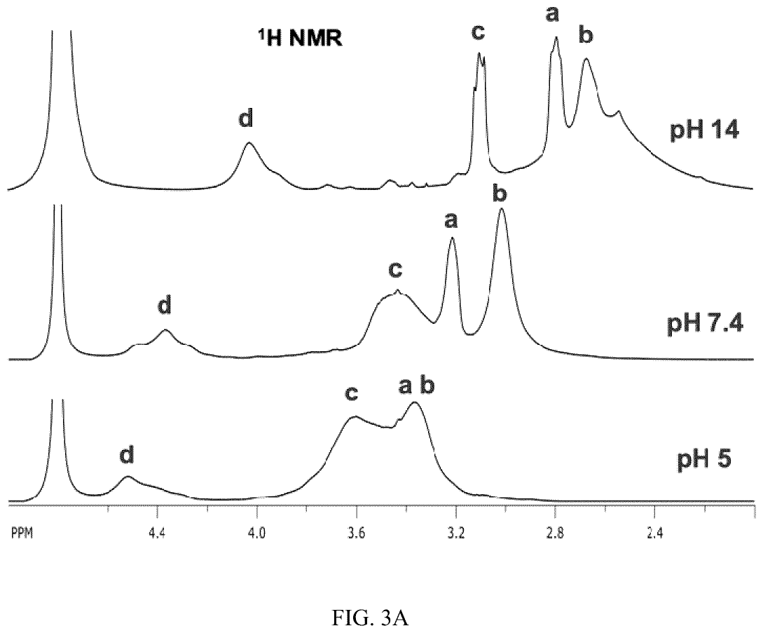

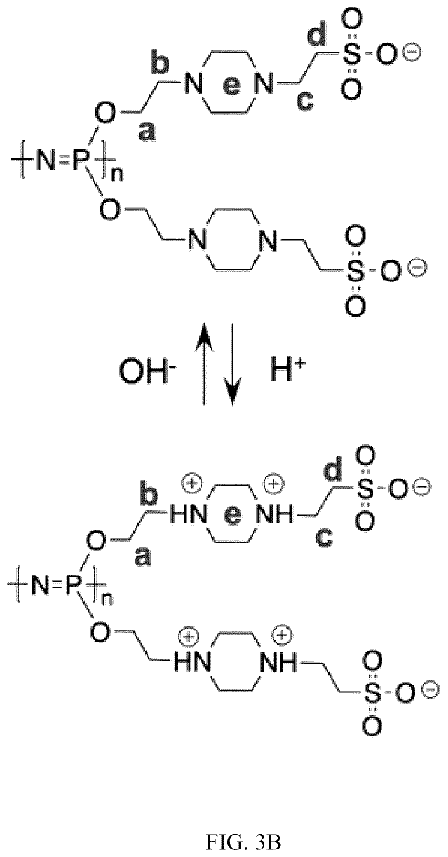

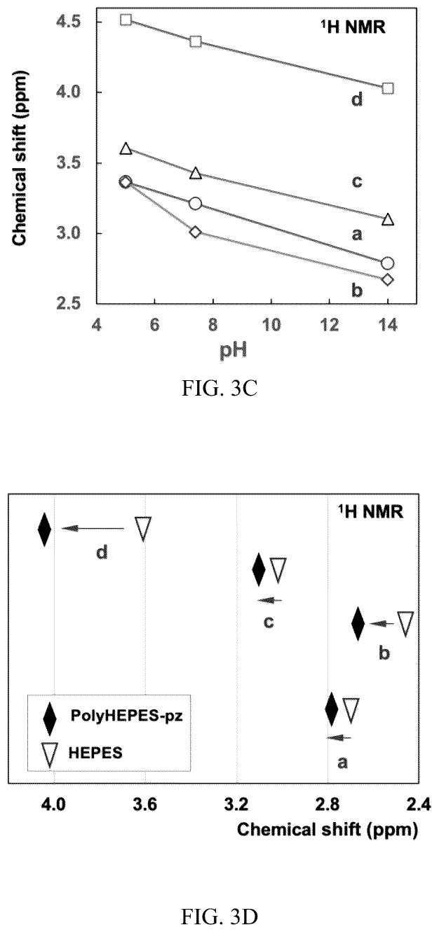

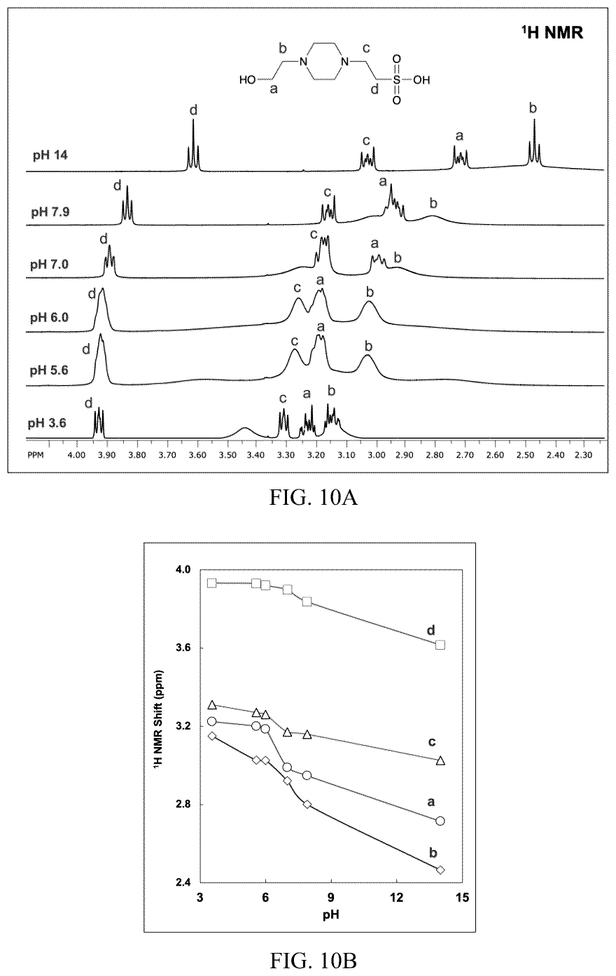

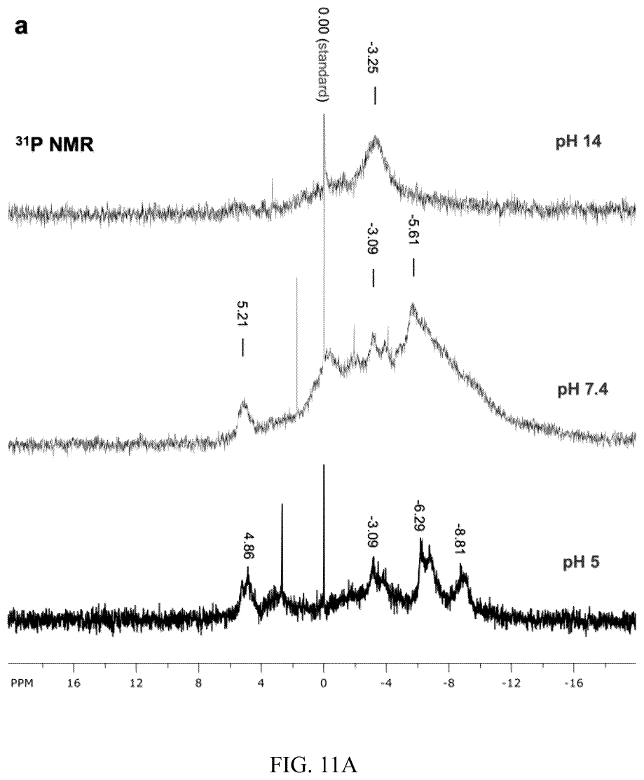

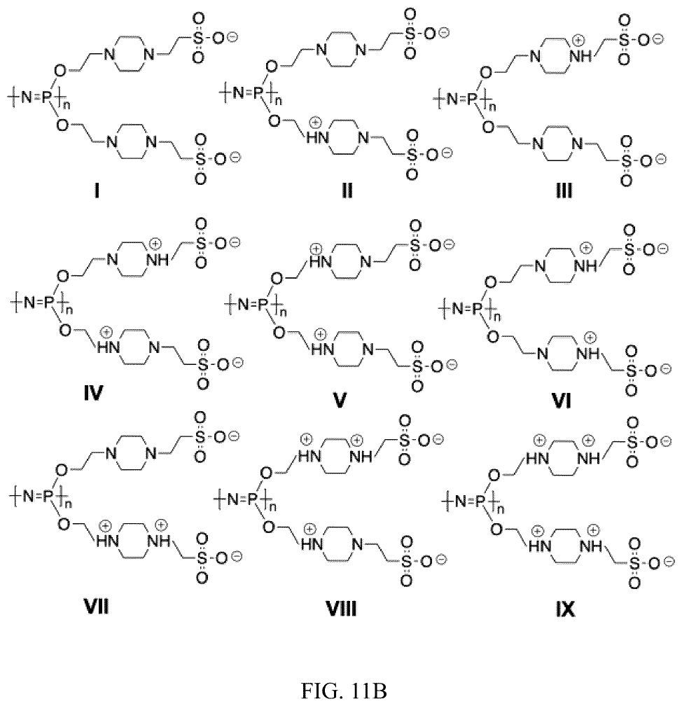

Structural Characteristics of PolyHEPES-pz and the Effect of its Ionization States. Structure and composition of polyHEPES-pz was analyzed by ¹³C, ¹H, and ³¹P NMR spectroscopy. ¹³C NMR spectrum of the polymer displays chemical shifts that are fully consistent with the expected structure of HEPES-containing homopolymer (FIG. 8). ¹H NMR spectra of both polyHEPES-pz and HEPES reveal a strong pH-dependence (FIGS. 3A, 9 and 10). This phenomenon is well-documented for HEPES and is explained by the deprotonation of piperazine moieties (FIG. 3B) in a basic environment, which causes shielding of non-labile hydrogen atoms. The addition of base causes the upfield shifts for four ¹H NMR peaks, which can be seen both for the HEPES and its macromolecular version (FIGS. 3C and 10b). However, the shifts are more pronounced for polyHEPES-pz. (FIG. 3D). This observation is in line with well-known fact that ionizable groups in the polymer do not deprotonate with the same strength as their monofunctional counterparts. ¹H NMR analysis of polyHEPES-pz also confirms the absence of potential impurities, such as DDTMA, which demonstrates the successful deprotection of HEPES side groups.

Strong pH-dependence is also observed for ³¹P NMR spectra, which show multiple peaks for neutral and acidic solutions (FIG. 11A). It is well-known that ³¹P NMR chemical shifts of polyphosphazenes are acutely sensitive to the type of side groups linked to the skeletal phosphorus atoms. For polyHEPES-pz, protonation of nitrogen atoms in side groups of the polymer can create up to nine distinct ionization patterns of repeat units (FIG. 11B). This may explain the complexity of ³¹P NMR spectra and their pH-dependent character. The presence of stable intermolecular interactions in HEPES solutions, which has been reported and attributed by the formation of strong hydrogen bonds can also be a factor. It is important, however, that the number of peaks decreases with the rise in pH and spectra obtained under strongly basic conditions show a single broad peak, which is consistent with polyHEPES-pz structure and the expected deprotonation pattern.

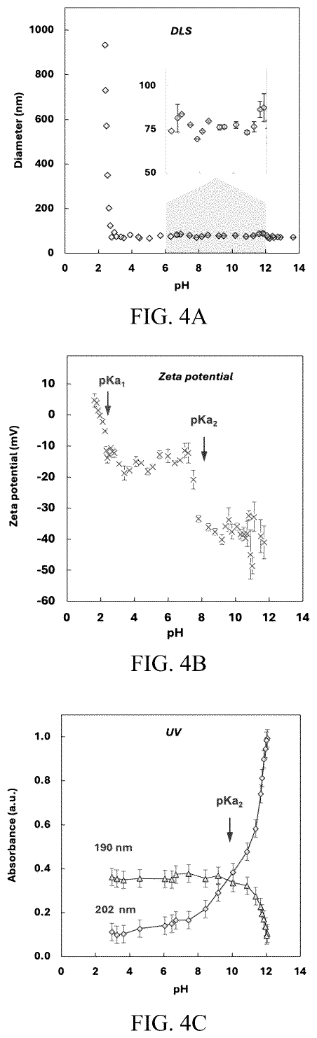

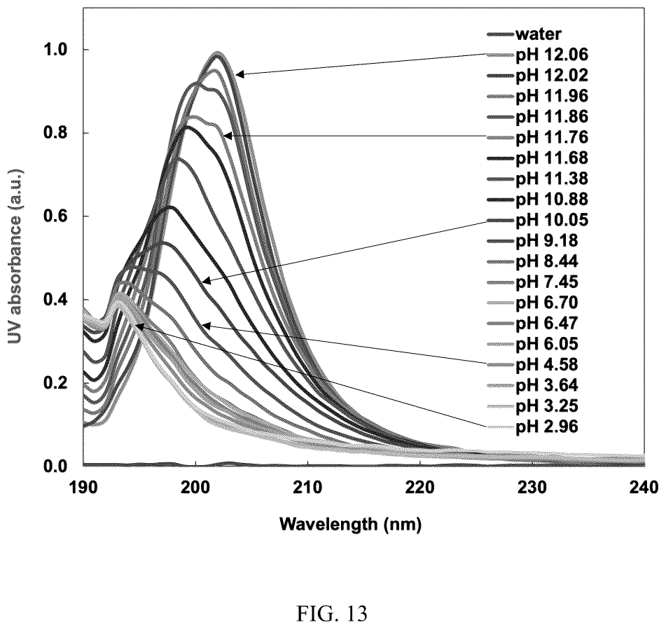

Solution Behavior. The behavior of a zwitterionic polymer in aqueous solutions is largely defined by environmental factors with pH, ionic strength and temperature playing a critical role in its solubility and conformational transitions. The complex patterns of poly HEPES-pz ionization states uncovered by ¹H and ³¹P NMR methods dictate the need for an in-depth physico-chemical investigation of its solution behavior. DLS studies of polyHEPES-pz in neutral, acidic, and basic solutions reveal that the polymer retains solubility and is essentially agglomerate-free over a broad pH range (FIG. 4A). The only exception—phase separation observed in highly acidic formulations, below pH 3. This suggests that the protonation of piperazine rings alone is not sufficient to prevent interchain association and the dissociation of acidic moieties remains critical for retaining polymer solubility. The phase transition pH correlates well with the reported dissociation constant for the sulfonic acid functionality of HEPES-pKₐ₁=2.9. It also corresponds to electroneutrality and first equivalent point determined by zeta potential titration of the polymer with hydrochloric acid (FIG. 4B). The second equivalence points on the same curve (at approximately pH 8) matches the dissociation constant of amines of the piperazine ring (pKₐ₂=7.7). The dissociation constant was also estimated by spectrophotometrical titration. UV spectra of poly HEPES-pz show that the protonation of a piperazine ring results in a pronounced decrease in the intensity of the main UV peak of the polymer and its strong hypsochromic shift (FIG. 13). Peak positions in most acidic and basic solutions (202 nm at pH 12 and 190) nm at pH 3) can be exploited to monitor UV absorbance values over the whole pH range and their intersection point utilized to estimate the dissociation constant (FIG. 4C). The pKₐ₂ value assessed by this technique was somewhat higher (pKₐ₂=9.1) than that obtained by zeta potential titration, which may be due to the technical limitations in the estimation of peak maximum absorbance at the lowest wavelength (extreme acidic conditions). However, this may also reflect the decrease in the deprotonation constant for a macromolecular version of the HEPES, which is consistent with variations in chemical shifts in NMR spectra for the polymer and its low-molar mass analog, which were discussed above. The shift in the pKₐ towards higher values may also be expected based on the well-known dependence of the dissociation constant of polyions on the degree of dissociation. Regardless, both methods confirm strong pH sensitivity of polyHEPES-pz and the results align well with the ampholytic character of the side group.

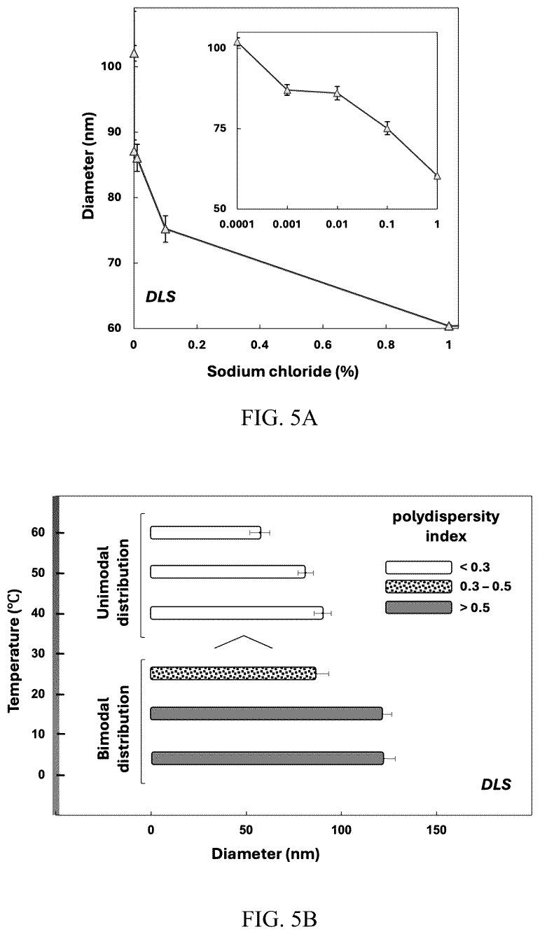

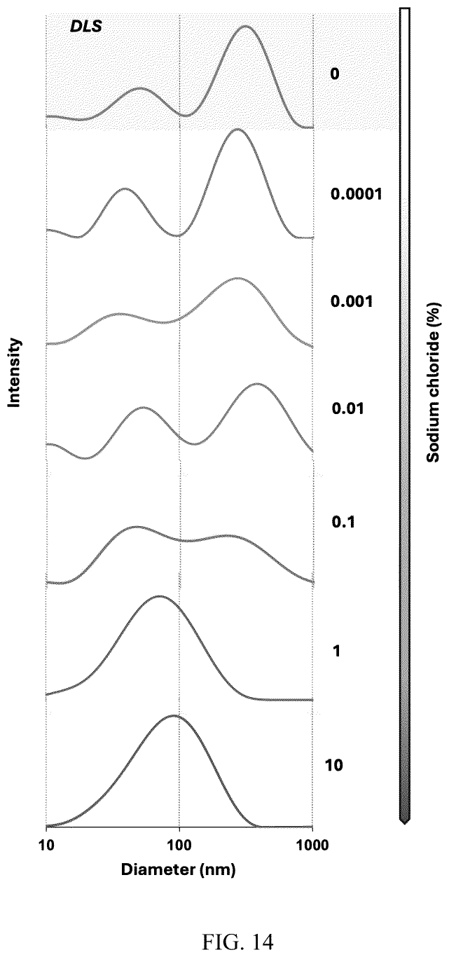

While all synthetic polyions show some sensitivity to the ionic strength of the solution, the response of zwitterionic polymers to the presence of salts is strikingly different from polyelectrolytes. Whereas most polyanions or polycations demonstrate the “salting-out” effect—decrease in solubility and eventual phase separation upon addition of salt, polyzwitterions display improved solubility at higher ionic strength, the so-called “salting-in” effect. Both phenomena result from screening of inter- and intrachain interactions by small ions. In case of polyelectrolytes, such screening affects repulsive interactions of similar charges on polyions leading to an improved solvation of macromolecular chains. In contrast, zwitterionic polymers tend to demonstrate attractive interactions between chains, which can be effectively shielded at a higher salt concentration. FIGS. 5A and 14 display a well-pronounced “salting-in” effect for polyHEPES-pz in the presence of sodium chloride, which is in line with the zwitterionic nature of this polymer.

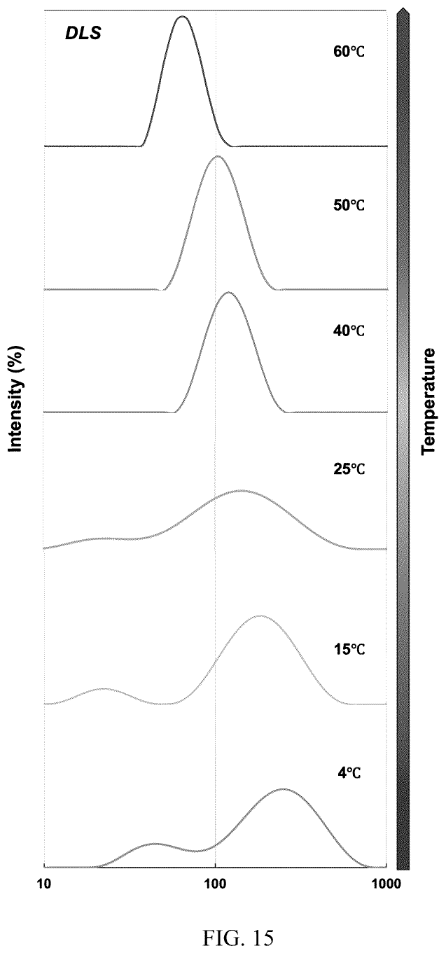

DLS studies of polyHEPES-pz in salt-free aqueous solutions reveal a noticeable thermal response (FIGS. 5B and 15). When dissolved in deionized water in the absence of salts, the presence of submicron agglomerates in samples is evident at and below 25° C. Heating of samples above those temperatures results in a complete elimination of submicron particulates and a gradual decrease in both z-average hydrodynamic diameter and polydispersity indexes. Such thermal response suggests that poly HEPES-pz shows an upper critical solution temperature (UCST) behavior. The findings are in line with well-documented behavior of zwitterionic polymers, which display attractive ionic interactions in low salt solutions. This leads to their precipitation or changes in morphology at lower temperatures and entropy driven solubility above the UCST. Both, the “salting in” and UCST-type behavior of poly HEPES-pz described above indicate the importance of attractive interchain interactions in the system and emphasize the ampholytic character of this polymer.

Hydrolytic Degradability of PolyHEPES-pz. An important prerequisite for the use of high molar mass polymers as injectables or bioerodible coatings is the development of their elimination pathways from the body. Non-biodegradable macromolecules are predominantly cleared through the glomerular filtration function of kidneys and the elimination of PEG is generally limited to those with the hydrodynamic radius of the renal threshold—5 nm. This roughly corresponds to synthetic polymers with molar masses ranging between 30 and 50 kDa. Such molar mass restrictions can reduce biological functionality of polymers and stealth characteristics, in particular. To eliminate this limitation, injectable macromolecules are designed to degrade into smaller fragments and physiologically benign products. One of the important rationales for designing macromolecular version of HEPES around inorganic phosphorus-nitrogen backbone is the ability of some polyphosphazenes to undergo hydrolytic breakdown in aqueous solutions. For those polyphosphazenes, degradation products typically include the released side group and the ammonium phosphate from the backbone. To that end, clinical safety of HEPES mentioned above can provide a key advantage. Therefore, polyHEPES-pz was investigated for the ability to undergo a hydrolytic breakdown in aqueous solutions.

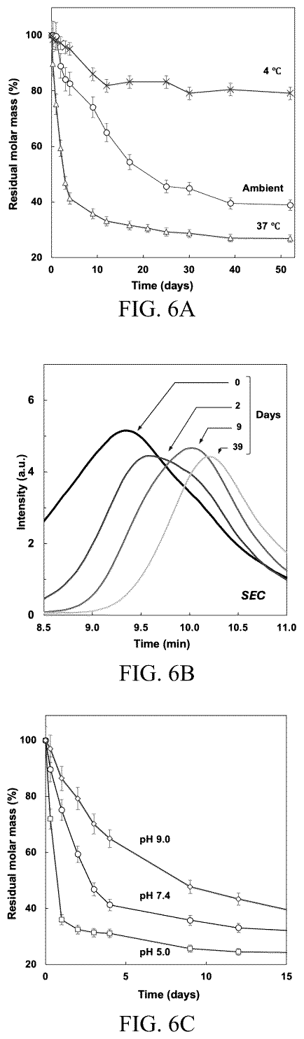

Time-dependent studies of polyHEPES-pz solutions by SEC reveal a gradual shift in the macromolecular peak towards lower molar masses (FIGS. 6A, 6B). Degradation plots display a pronounced temperature dependence. The half-life of the polymer at 37° C. is estimated at three days but is dramatically slower under refrigeration—only 20% molar mass loss observed after 30 days of incubation at 4° C. (FIG. 6A). Such thermoresponsive behavior provides for a potentially tunable degradation profiles under physiological conditions and adequate shelf-life upon refrigeration or freezing.

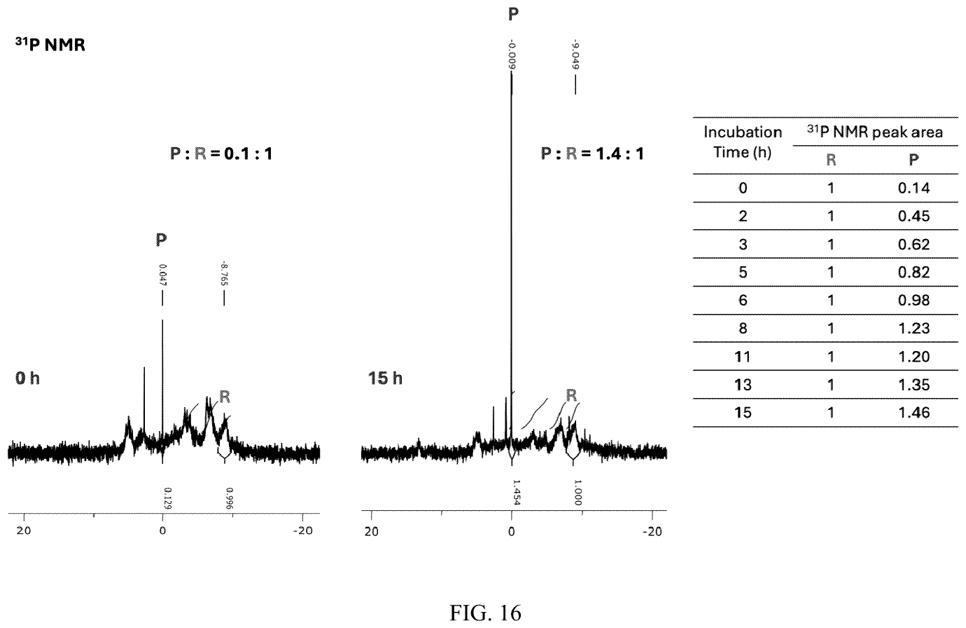

The degradation rate is also highly sensitive to the pH of solution. In fact, basic environment causes a dramatic inhibition of polymer degradation—a stunning ten-fold increase in half-life at pH 9 compared to the acidic environment. pH 5 (FIG. 6C). This pH-dependence of degradation rates is characteristic of the acid catalyzed mechanism of polyphosphazene hydrolysis and can be used as an additional tool for modulating stability of the formulation. ³¹P NMR investigation of the system under acidic conditions shows the formation of a phosphate peak at 0 ppm (designated as “P”), which is typically attributed to phosphates (FIG. 16). The ratio of the phosphate (“P”) and a representative macromolecular peak (“R”: −8 ppm) continues to grow as the degradation progressed, once again, indicating that the degradation process follows a typical polyphosphazene mechanism.

Biologically Relevant Properties. The absolute majority of stealth polymers are designed for use in applications, which entail their contact with blood or its components. Therefore, hemocompatibility is one of the critical criteria for the selection of polymer as a carrier for drug delivery formulations or antifouling coating material. To that end, the hemolytic activity of polyHEPES-pz was evaluated using human red blood cells. The results were compared with PEG and polyphosphazene polyanion poly[di(carboxylatophenoxy)phosphazene](PCPP), which showed excellent safety profile in clinical studies. Polyethyleneimine (PEI)—a cationic polyelectrolyte, which has been one of the most frequent choices for gene delivery, was employed as a positive control. As expected. under the conditions of the experiment, cationic PEI causes the lysis of almost 50% of cells. Hemolytic activity of polyHEPES-pz (0.2% hemolysis) is negligible and the same or even lower than that of PEG and PCPP. These results demonstrate excellent in vitro hemocompatibility of the HEPES-based zwitterionic polymer.

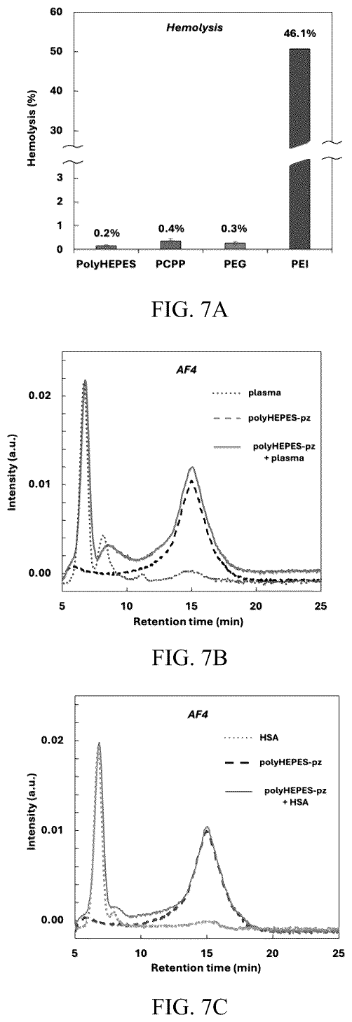

Engineering molecular systems for attaining effective stealth properties involves elimination of protein-attractive binding sites on the polymer chain and enhancement of its protein-repulsive forces. The initial assessment of bioinert materials encompasses their evaluation for possible interactions with blood plasma and its components. Asymmetric flow field flow fractionation (AF4)—a gentle characterization technique, which separates analytes based on their size in the absence of a stationary phase, has been recently suggested as a method for studying labile macromolecular interactions and the analysis of complex body fluids, such as blood plasma. In particular, the AF4 method was successfully employed to demonstrate the formation of protein corona around PCPP macromolecule upon its exposure to diluted human blood plasma. A typical AF4 fractogram of human plasma displays two main peaks at approximately 7 and 9 min (FIG. 7B), which were assigned to human serum albumin (HSA) and immunoglobulin G, correspondingly. Analysis of polyHEPES-pz with AF4 technique reveals a single peak at approximately 15 min. which remains practically unchanged upon addition of the diluted plasma. Furthermore, in the fractogram of polyHEPES-pz—plasma mixture, the HSA and the immunoglobulin G peaks retain their position and peak areas, although the 9 min peak shows some broadening compared to that in the fractogram of the plasma. This is most likely due to some superposition with a relatively broad polymer trace as polyHEPES had to be analyzed at a high injection size to compensate for its low extinction coefficient. The results suggest the absence of interactions between polyHEPES-pz and blood plasma. The validity of this conclusion is supported by previous findings, which demonstrated a successful AF4 detection of polymer-bound protein corona under the same conditions, but for a different system-PCPP-plasma formulation. FIG. 7C shows a fractogram of polyHEPES-pz in the presence of the main plasma component—HSA, which also confirms the absence of interactions with this physiologically abundant protein. Taken together, the ability of polyHEPES-pz to withstand interactions with plasma proteins, its hemocompatibility and adequate hydrolytic degradation profiles, establish reasonable biophysical grounds for its further investigation as a PEG-alternative macromolecule.

Polyzwitterions emerged as one of the main focuses in a search for “PEG-alternative” macromolecules due to their remarkable protein repulsive properties and general biocompatibility. Although small zwitterionic molecules are abundant in life, strategies for the synthesis of macromolecules with zwitterionic groups rely mainly on bioinspiration, rather than on biomimicry. Contrary to polyampholytes, such as proteins and polynucleotides, polyzwitterions are not found in nature. HEPES—a widespread synthetic buffering agent with a physiologically useful pH range and proven clinical safety, presents an attractive building block for molecular engineering of biomedical polymers. Hybrid organic-inorganic polyphosphazene backbone, which can provide for hydrolytic degradability under physiological conditions, constitutes a convenient scaffold for a multimeric assembly of this zwitterionic molecule.

The successful realization of polyHEPES-pz synthetic pathway proves the applicability of an ion-based approach to the in-situ protection of molecules containing zwitterionic functionalities. It offers a simple and convenient route to a multimeric assembly of hundreds or thousands HEPES copies on a single polymer chain, depending on the molar mass of the original macromolecular precursor. The resulting polyHEPES-pz is a linear macromolecule with unimodal size distribution, which shows excellent solubility in aqueous solutions and displays typical polyampholytic behavior. The potential utility of polyphosphazene with zwitterionic pendant group for life sciences applications is validated by PEG-comparable hemocompatibility, its non-interactive profile and ability to undergo hydrolytic degradation at near physiological conditions.

The versatility of synthetic polyphosphazene methodology offers multiple opportunities for further structural diversification. Insertion of reactive functionalities by a macromolecular co-substitution route can provide convenient sites for covalent conjugation of protein or small molecule therapeutics to this polymer. The molecular size restrictions, which constitute a major limitation for the use of non-degradable PEGs with injectable formulations, are not expected to apply to a hydrolytically degradable polyHEPES-pz. Temperature and pH-dependence of polymer degradation can allow further tuning of in vivo performance and shelf-life optimization. It is envisioned, that polyHEPES-pz can be suitable for the development of advanced bioerodible coatings with antifouling characteristics.

Abbreviations. HEPES, 4-(2-hydroxyethyl)-1-piperazineethanesulfonic acid; TEA, triethylamine: DDTMA, dodecyltrimethyl ammonium bromide, DDTMA: PDCP, polydichlorophosphazene: polyHEPES-pz, polyphosphazene containing HEPES side groups: NMR, nuclear magnetic resonance: AF4, asymmetric flow field flow fractionation: SEC, size-exclusion chromatography, DLS, dynamic light scattering: cryo-EM, cryogenic electron microscopy: PCPP, poly[di(carboxylatophenoxy)phosphazene]: PEG, poly(ethylene glycol); PEI, polyethyleneimine: RBCs, human red blood cells: HSA, human serum albumin.

Although the present disclosure has been described with respect to one or more particular embodiments and/or examples, it will be understood that other embodiments and/or examples of the present disclosure may be made without departing from the scope of the present disclosure.

1. A polymer comprising the following structure: 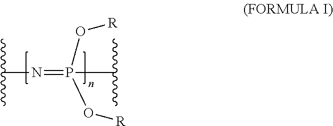
wherein
each R group may be the same or different,
each R group is individually a substituted or unsubstituted aliphatic group or substituted or unsubstituted aryl group, wherein at least one R group of the polymer is a zwitterionic group or capable of being ionized such that the R group is zwitterionic, and
n is 1 to 500,000.

2. The polymer according to claim 1, wherein the at least one R group has the following structure: 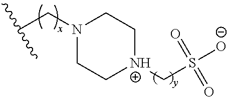
or a protonated or deprotonated form thereof, wherein x is 1 to 3 and y is 1 to 3.

3. The polymer according to claim 1, wherein the at least one R group is 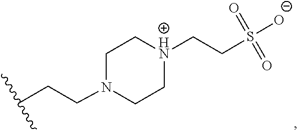
or a protonated or deprotonated form thereof.

4. The polymer according to claim 1, wherein 1 to all R groups are 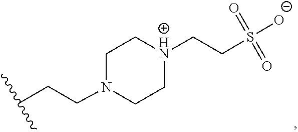
or a protonated or deprotonated form thereof.

5. The polymer according to claim 1, wherein the polymer is a copolymer comprising additional polymer units.

6. The polymer according to claim 5, wherein the copolymer is a block copolymer.

7. The polymer according to claim 5, wherein the copolymer is a random copolymer.

8. The polymer according to claim 1, wherein the R groups comprise one or more of the following substituents: carboxylic acid groups, carboxylate groups, sulfonic acid groups, sulfonate groups, hydroxyl groups, oxy groups, sulfhydryl groups, imidazolyl groups, phenolic groups, guanidyl groups, indolyl groups, ammonium groups, or amino groups.

9. The polymer according to claim 1, wherein R is

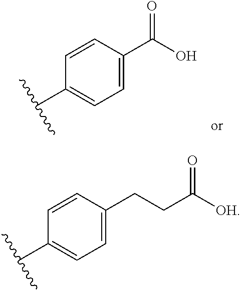 [SMILES: *c1ccc(C(=O)O)cc1 ; *c1ccc(CCC(=O)O)cc1]

10. The polymer according to claim 1, wherein the polymer has the following structure: 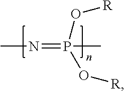
wherein every R group is: 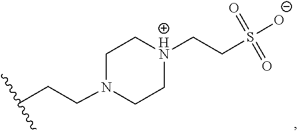
or a protonated or deprotonated form thereof, and n is 3 to 500,000.

11. The polymer according to claim 1, wherein the polymer has a molecular weight of 1 kDa to 1,000 kDa.

12. The composition comprising one or more polymers according to claim 1.

13. The composition according to claim 12, further comprising a pharmaceutically acceptable carrier.

14. The composition according to claim 12, further comprising one or more pharmaceutical agent.

15. The composition according to claim 14, wherein the pharmaceutical agent is chosen from proteins, antibodies, peptides, enzymes, nucleic acids, drugs, small molecules, siRNA, and the like and combinations thereof.

16. A method of delivering a pharmaceutical agent to a subject in need of treatment comprising administering a composition according to claim 14 to the subject.

17. A substrate wherein a polymer according to claim 1 is disposed thereon.

18. The substrate according to claim 17, wherein the substrate is a medical device or prosthetic.
